SEQUENCE LISTING (25 sequences)

SEQ ID NO: 1 - DNA, 3804 bp, Artificial Sequence
  vector genome hSyn.hUbe3a-1.GSco.4XmiRNA183.SV40
  repeat_region (1)..(130): ITR
  promoter (213)..(678): hSynapsin
  misc_feature (690)..(695): Kozak
  CDS (696)..(3257): hUBE3a-1
  misc_feature (779)..(779): mut
  misc_feature (824)..(824): mut
  misc_feature (1028)..(1028): mut
  misc_feature (1128)..(1128): mut
  misc_feature (1181)..(1181): mut
  misc_feature (1217)..(1217): mut
  misc_feature (1283)..(1283): mut SD/AS
  misc_feature (1715)..(1715): mut
  misc_feature (1721)..(1721): mut
  misc_feature (1766)..(1766): mut
  misc_feature (1805)..(1805): mut
  misc_feature (1814)..(1814): mut
  misc_feature (1991)..(1991): mut
  misc_feature (2063)..(2063): mut
  misc_feature (2111)..(2111): mut
  misc_feature (2294)..(2294): mut
  misc_feature (2439)..(2440): mut SD/AS
  misc_feature (2723)..(2723): mut
  misc_feature (2957)..(2957): mut
  misc_feature (3023)..(3023): mut
  misc_feature (3056)..(3056): mut SD/AS
  misc_feature (3152)..(3152): mut
  misc_feature (3161)..(3161): mut
  misc_feature (3230)..(3230): mut
  misc_feature (3264)..(3285): miRNA183
  misc_feature (3318)..(3311): miRNA183
  misc_feature (3318)..(3339): miRNA183
  misc_feature (3346)..(3367): miRNA183
  polyA_signal (3379)..(3610): SV40 polyA
  repeat_region (3675)..(3804): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatgaagagggcagcagcaaagcacctgatcgagagatactatcaccagctgaccgagggatgcggaaacgaggcatgtacaaacgagttctgcgcctcctgtcccacctttctgaggatggataacaacgccgccgccatcaaggccctggagctgtacaagatcaacgccaagctgtgcgacccccaccctagcaagaagggcgccagctccgcctatctggagaactccaagggcgcccctaacaatagctgttccgagatcaagatgaataagaagggcgcccggatcgatttcaaggacgtgacctacctgacagaggagaaggtgtacgagatcctggagctgtgccgggagagagaggattacagcccactgatcagagtgatcggcagagtgttctctagcgccgaggccctggtgcagtcctttagaaaggtgaagcagcacacaaaggaggagctgaagtctctgcaggccaaggacgaggacaaggacgaggacgagaaggagaaggcagcctgttctgccgcagcaatggaggaggacagcgaggcatcctctagccggatcggcgattcctctcaaggcgacaacaatctgcagaagctgggccccgacgacgtgagcgtggatatcgacgccatccggagagtgtacacaagactgctgagcaacgagaagatcgagaccgccttcctgaacgccctggtgtatctgagccctaatgtggagtgcgatctgacctaccacaacgtgtactcccgggacccaaactacctgaatctgttcatcatcgtgatggagaacagaaatctgcactcccccgagtatctggagatggccctgcctctgttttgtaaggccatgtccaagctgcctctggcagcacagggcaagctgatcaggctgtggtctaagtacaacgccgatcagatcaggcgcatgatggagaccttccagcagctgatcacatacaaagtgatctctaacgagtttaatagccgcaacctggtgaacgacgacgacgccatcgtggccgcctctaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtactccgagaggcgcatcaccgtgctgtattctctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtctaaggagttctttcagctggtggtggaggagatcttcaaccccgatatcggcatgtttacctacgacgagagcacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgactcccaccccgtgctgtaccagtctctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaatagaaaggagttcgtgaacctgtacagcgattatatcctgaataagtccgtggagaagcagttcaaggcctttaggcgcggcttccacatggtgaccaacgagagccctctgaagtatctgtttaggccagaggagatcgagctgctgatctgcggctcccgcaatctggactttcaggccctggaggagaccacagagtacgacggcggctataccagggactctgtgctgatccgcgagttctgggagatcgtgcacagctttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccagtgaattctaccagtgccataggatagtgaattctaccagtgccatacacgtgagtgaattctaccagtgccatagcatgcagtgaattctaccagtgccataggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - protein, 852 aa, Artificial Sequence
  Synthetic Construct
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 3 - DNA, 3700 bp, Artificial Sequence
  vector genome hSyn.hUbe3a-1.GSco.SV40
  repeat_region (1)..(130): ITR
  promoter (213)..(678): hSynapsin promoter
  misc_feature (690)..(695): Kozak
  CDS (696)..(3257): hUBE3a-1
  misc_feature (779)..(779): mut
  misc_feature (824)..(824): mut
  misc_feature (1028)..(1028): mut
  misc_feature (1181)..(1181): mut
  misc_feature (1193)..(1193): mut
  misc_feature (1217)..(1217): mut
  misc_feature (1283)..(1283): mut SD/AS
  misc_feature (1715)..(1715): mut
  misc_feature (1721)..(1721): mut
  misc_feature (1766)..(1766): mut
  misc_feature (1805)..(1805): mut
  misc_feature (1814)..(1814): mut
  misc_feature (1991)..(1991): mut
  misc_feature (2063)..(2063): mut
  misc_feature (2111)..(2111): mut
  misc_feature (2294)..(2294): mut
  misc_feature (2439)..(2440): mut SD/AS
  misc_feature (2723)..(2723): mut
  misc_feature (2957)..(2957): mut
  misc_feature (3023)..(3023): mut
  misc_feature (3056)..(3056): mut SD/AS
  misc_feature (3152)..(3152): mut
  misc_feature (3161)..(3161): mut
  misc_feature (3230)..(3230): mut
  polyA_signal (3275)..(3700): SV40 polyA
  repeat_region (3571)..(3700): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatgaagagggcagcagcaaagcacctgatcgagagatactatcaccagctgaccgagggatgcggaaacgaggcatgtacaaacgagttctgcgcctcctgtcccacctttctgaggatggataacaacgccgccgccatcaaggccctggagctgtacaagatcaacgccaagctgtgcgacccccaccctagcaagaagggcgccagctccgcctatctggagaactccaagggcgcccctaacaatagctgttccgagatcaagatgaataagaagggcgcccggatcgatttcaaggacgtgacctacctgacagaggagaaggtgtacgagatcctggagctgtgccgggagagagaggattacagcccactgatcagagtgatcggcagagtgttctctagcgccgaggccctggtgcagtcctttagaaaggtgaagcagcacacaaaggaggagctgaagtctctgcaggccaaggacgaggacaaggacgaggacgagaaggagaaggcagcctgttctgccgcagcaatggaggaggacagcgaggcatcctctagccggatcggcgattcctctcaaggcgacaacaatctgcagaagctgggccccgacgacgtgagcgtggatatcgacgccatccggagagtgtacacaagactgctgagcaacgagaagatcgagaccgccttcctgaacgccctggtgtatctgagccctaatgtggagtgcgatctgacctaccacaacgtgtactcccgggacccaaactacctgaatctgttcatcatcgtgatggagaacagaaatctgcactcccccgagtatctggagatggccctgcctctgttttgtaaggccatgtccaagctgcctctggcagcacagggcaagctgatcaggctgtggtctaagtacaacgccgatcagatcaggcgcatgatggagaccttccagcagctgatcacatacaaagtgatctctaacgagtttaatagccgcaacctggtgaacgacgacgacgccatcgtggccgcctctaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtactccgagaggcgcatcaccgtgctgtattctctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtctaaggagttctttcagctggtggtggaggagatcttcaaccccgatatcggcatgtttacctacgacgagagcacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgactcccaccccgtgctgtaccagtctctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaatagaaaggagttcgtgaacctgtacagcgattatatcctgaataagtccgtggagaagcagttcaaggcctttaggcgcggcttccacatggtgaccaacgagagccctctgaagtatctgtttaggccagaggagatcgagctgctgatctgcggctcccgcaatctggactttcaggccctggaggagaccacagagtacgacggcggctataccagggactctgtgctgatccgcgagttctgggagatcgtgcacagctttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - protein, 852 aa, Artificial Sequence; identical to SEQ ID NO: 2
  Synthetic Construct

SEQ ID NO: 5 - DNA, 3873 bp, Artificial Sequence
  vector genome hSyn.hUbe3a-2.GSco.4XmiRNA183.SV40
  repeat_region (1)..(130): ITR
  promoter (213)..(678): hSynapsin promoter
  misc_feature (690)..(695): Kozak
  CDS (696)..(3326): hUBE3a-2
  misc_feature (848)..(848): mut
  misc_feature (893)..(893): mut
  misc_feature (1097)..(1097): mut
  misc_feature (1250)..(1250): mut
  misc_feature (1262)..(1262): mut
  misc_feature (1286)..(1286): mut
  misc_feature (1352)..(1352): mut SD/AS
  misc_feature (1784)..(1784): mut
  misc_feature (1790)..(1790): mut
  misc_feature (1835)..(1835): mut
  misc_feature (1874)..(1874): mut
  misc_feature (1883)..(1883): mut
  misc_feature (2060)..(2060): mut
  misc_feature (2132)..(2132): mut
  misc_feature (2180)..(2180): mut
  misc_feature (2363)..(2363): mut
  misc_feature (2508)..(2508): mut SD/AS
  misc_feature (2792)..(2792): mut
  misc_feature (3026)..(3026): mut
  misc_feature (3092)..(3092): mut
  misc_feature (3125)..(3125): mut SD/AS
  misc_feature (3221)..(3221): mut
  misc_feature (3230)..(3230): mut
  misc_feature (3299)..(3299): mut
  misc_feature (3333)..(3354): miRNA183
  misc_feature (3359)..(3380): miRNA183
  misc_feature (3387)..(3408): miRNA183
  misc_feature (3415)..(3436): miRNA183
  polyA_signal (3448)..(3679): SV40 polyA
  repeat_region (3744)..(3873): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggagaagctgcaccagtgctactggaagtctggcgagcctcagagcgacgatatcgaggcaagcaggatgaagagagcagcagccaagcacctgatcgagcggtactatcaccagctgaccgagggctgcggaaacgaggcctgtacaaacgagttctgcgcctcctgtccaacctttctgagaatggataacaacgccgccgccatcaaggccctggagctgtacaagatcaacgccaagctgtgcgacccccacccttctaagaagggcgccagctccgcctatctggagaacagcaagggcgcccccaacaatagctgttccgagatcaagatgaataagaagggcgccaggatcgatttcaaggacgtgacctacctgacagaggagaaggtgtacgagatcctggagctgtgccgggagagagaggattactcccctctgatcagagtgatcggacgcgtgttctctagcgccgaggccctggtgcagagctttcgcaaggtgaagcagcacacaaaggaggagctgaagtccctgcaggccaaggacgaggacaaggacgaggacgagaaggagaaggcagcttgttccgccgcagcaatggaggaggactctgaggcctcctctagcaggatcggcgattcctctcaaggcgacaacaatctgcagaagctgggccccgacgacgtgagcgtggatatcgacgccatccggagagtgtacacacgcctgctgtctaacgagaagatcgagaccgccttcctgaacgccctggtgtatctgtctcctaatgtggagtgcgatctgacctaccacaacgtgtacagccgggacccaaactacctgaatctgttcatcatcgtgatggagaacagaaatctgcactctcccgagtatctggagatggccctgcctctgttttgtaaggccatgagcaagctgccactggcagcacagggcaagctgatcaggctgtggtccaagtacaacgccgatcagatcaggcgcatgatggagaccttccagcagctgatcacatataaagtgatctccaacgagtttaattctagaaacctggtgaacgacgacgacgccatcgtggccgccagcaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtacagcgagaggcgcatcaccgtgctgtattccctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtccaaggagttctttcagctggtggtggaggagatcttcaaccccgatatcggcatgtttacctacgacgagtctacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgacagccaccccgtgctgtaccagtccctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaataggaaggagttcgtgaacctgtactctgattatatcctgaataagagcgtggagaagcagttcaaggcctttaggcgcggcttccacatggtgaccaacgagtctcctctgaagtatctgtttaggccagaggagatcgagctgctgatctgcggcagccgcaatctggactttcaggccctggaggagaccacagagtacgacggcggctatacccgggactccgtgctgatcagagagttctgggagatcgtgcactcttttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccagtgaattctaccagtgccataggatagtgaattctaccagtgccatacacgtgagtgaattctaccagtgccatagcatgcagtgaattctaccagtgccataggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - protein, 875 aa, Artificial Sequence
  Synthetic Construct
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 7 - DNA, 3769 bp, Artificial Sequence
  vector genome hSyn.hUbe3a-2.GSco.SV40
  repeat_region (1)..(130): ITR
  promoter (213)..(678): hSynapsin promoter
  misc_feature (690)..(695): Kozak
  CDS (696)..(3326): hUBE3a-2
  misc_feature (848)..(848): mut
  misc_feature (893)..(893): mut
  misc_feature (1097)..(1097): mut
  misc_feature (1250)..(1250): mut
  misc_feature (1262)..(1262): mut
  misc_feature (1286)..(1286): mut
  misc_feature (1352)..(1352): mut SD/AS
  misc_feature (1784)..(1784): mut
  misc_feature (1790)..(1790): mut
  misc_feature (1835)..(1835): mut
  misc_feature (1874)..(1874): mut
  misc_feature (1883)..(1883): mut
  misc_feature (2060)..(2060): mut
  misc_feature (2132)..(2132): mut
  misc_feature (2180)..(2180): mut
  misc_feature (2363)..(2363): mut
  misc_feature (2508)..(2509): mut SD/AS
  misc_feature (2792)..(2792): mut
  misc_feature (3026)..(3026): mut
  misc_feature (3092)..(3092): mut
  misc_feature (3125)..(3125): mut SD/AS
  misc_feature (3221)..(3221): mut
  misc_feature (3230)..(3230): mut
  misc_feature (3299)..(3299): mut
  polyA_signal (3344)..(3757): SV40 polyA
  repeat_region (3640)..(3769): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggagaagctgcaccagtgctactggaagtctggcgagcctcagagcgacgatatcgaggcaagcaggatgaagagagcagcagccaagcacctgatcgagcggtactatcaccagctgaccgagggctgcggaaacgaggcctgtacaaacgagttctgcgcctcctgtccaacctttctgagaatggataacaacgccgccgccatcaaggccctggagctgtacaagatcaacgccaagctgtgcgacccccacccttctaagaagggcgccagctccgcctatctggagaacagcaagggcgcccccaacaatagctgttccgagatcaagatgaataagaagggcgccaggatcgatttcaaggacgtgacctacctgacagaggagaaggtgtacgagatcctggagctgtgccgggagagagaggattactcccctctgatcagagtgatcggacgcgtgttctctagcgccgaggccctggtgcagagctttcgcaaggtgaagcagcacacaaaggaggagctgaagtccctgcaggccaaggacgaggacaaggacgaggacgagaaggagaaggcagcttgttccgccgcagcaatggaggaggactctgaggcctcctctagcaggatcggcgattcctctcaaggcgacaacaatctgcagaagctgggccccgacgacgtgagcgtggatatcgacgccatccggagagtgtacacacgcctgctgtctaacgagaagatcgagaccgccttcctgaacgccctggtgtatctgtctcctaatgtggagtgcgatctgacctaccacaacgtgtacagccgggacccaaactacctgaatctgttcatcatcgtgatggagaacagaaatctgcactctcccgagtatctggagatggccctgcctctgttttgtaaggccatgagcaagctgccactggcagcacagggcaagctgatcaggctgtggtccaagtacaacgccgatcagatcaggcgcatgatggagaccttccagcagctgatcacatataaagtgatctccaacgagtttaattctagaaacctggtgaacgacgacgacgccatcgtggccgccagcaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtacagcgagaggcgcatcaccgtgctgtattccctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtccaaggagttctttcagctggtggtggaggagatcttcaaccccgatatcggcatgtttacctacgacgagtctacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgacagccaccccgtgctgtaccagtccctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaataggaaggagttcgtgaacctgtactctgattatatcctgaataagagcgtggagaagcagttcaaggcctttaggcgcggcttccacatggtgaccaacgagtctcctctgaagtatctgtttaggccagaggagatcgagctgctgatctgcggcagccgcaatctggactttcaggccctggaggagaccacagagtacgacggcggctatacccgggactccgtgctgatcagagagttctgggagatcgtgcactcttttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - protein, 875 aa, Artificial Sequence; identical to SEQ ID NO: 6
  Synthetic Construct

SEQ ID NO: 9 - DNA, 2562 bp, Artificial Sequence
  nucleic acid sequence hUbe31a-1
atgaagagggcagcagcaaagcacctgatcgagagatactatcaccagctgaccgagggatgcggaaacgaggcatgtacaaacgagttctgcgcctcctgtcccacctttctgaggatggataacaacgccgccgccatcaaggccctggagctgtacaagatcaacgccaagctgtgcgacccccaccctagcaagaagggcgccagctccgcctatctggagaactccaagggcgcccctaacaatagctgttccgagatcaagatgaataagaagggcgcccggatcgatttcaaggacgtgacctacctgacagaggagaaggtgtacgagatcctggagctgtgccgggagagagaggattacagcccactgatcagagtgatcggcagagtgttctctagcgccgaggccctggtgcagtcctttagaaaggtgaagcagcacacaaaggaggagctgaagtctctgcaggccaaggacgaggacaaggacgaggacgagaaggagaaggcagcctgttctgccgcagcaatggaggaggacagcgaggcatcctctagccggatcggcgattcctctcaaggcgacaacaatctgcagaagctgggccccgacgacgtgagcgtggatatcgacgccatccggagagtgtacacaagactgctgagcaacgagaagatcgagaccgccttcctgaacgccctggtgtatctgagccctaatgtggagtgcgatctgacctaccacaacgtgtactcccgggacccaaactacctgaatctgttcatcatcgtgatggagaacagaaatctgcactcccccgagtatctggagatggccctgcctctgttttgtaaggccatgtccaagctgcctctggcagcacagggcaagctgatcaggctgtggtctaagtacaacgccgatcagatcaggcgcatgatggagaccttccagcagctgatcacatacaaagtgatctctaacgagtttaatagccgcaacctggtgaacgacgacgacgccatcgtggccgcctctaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtactccgagaggcgcatcaccgtgctgtattctctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtctaaggagttctttcagctggtggtggaggagatcttcaaccccgatatcggcatgtttacctacgacgagagcacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgactcccaccccgtgctgtaccagtctctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaatagaaaggagttcgtgaacctgtacagcgattatatcctgaataagtccgtggagaagcagttcaaggcctttaggcgcggcttccacatggtgaccaacgagagccctctgaagtatctgtttaggccagaggagatcgagctgctgatctgcggctcccgcaatctggactttcaggccctggaggagaccacagagtacgacggcggctataccagggactctgtgctgatccgcgagttctgggagatcgtgcacagctttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatga

SEQ ID NO: 10 - DNA, 2631 bp, Artificial Sequence
  nucleic acid sequence hUbe3a-2
atggagaagctgcaccagtgctactggaagtctggcgagcctcagagcgacgatatcgaggcaagcaggatgaagagagcagcagccaagcacctgatcgagcggtactatcaccagctgaccgagggctgcggaaacgaggcctgtacaaacgagttctgcgcctcctgtccaacctttctgagaatggataacaacgccgccgccatcaaggccctggagctgtacaagatcaacgccaagctgtgcgacccccacccttctaagaagggcgccagctccgcctatctggagaacagcaagggcgcccccaacaatagctgttccgagatcaagatgaataagaagggcgccaggatcgatttcaaggacgtgacctacctgacagaggagaaggtgtacgagatcctggagctgtgccgggagagagaggattactcccctctgatcagagtgatcggacgcgtgttctctagcgccgaggccctggtgcagagctttcgcaaggtgaagcagcacacaaaggaggagctgaagtccctgcaggccaaggacgaggacaaggacgaggacgagaaggagaaggcagcttgttccgccgcagcaatggaggaggactctgaggcctcctctagcaggatcggcgattcctctcaaggcgacaacaatctgcagaagctgggccccgacgacgtgagcgtggatatcgacgccatccggagagtgtacacacgcctgctgtctaacgagaagatcgagaccgccttcctgaacgccctggtgtatctgtctcctaatgtggagtgcgatctgacctaccacaacgtgtacagccgggacccaaactacctgaatctgttcatcatcgtgatggagaacagaaatctgcactctcccgagtatctggagatggccctgcctctgttttgtaaggccatgagcaagctgccactggcagcacagggcaagctgatcaggctgtggtccaagtacaacgccgatcagatcaggcgcatgatggagaccttccagcagctgatcacatataaagtgatctccaacgagtttaattctagaaacctggtgaacgacgacgacgccatcgtggccgccagcaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtacagcgagaggcgcatcaccgtgctgtattccctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtccaaggagttctttcagctggtggtggaggagatcttcaaccccgatatcggcatgtttacctacgacgagtctacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgacagccaccccgtgctgtaccagtccctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaataggaaggagttcgtgaacctgtactctgattatatcctgaataagagcgtggagaagcagttcaaggcctttaggcgcggcttccacatggtgaccaacgagtctcctctgaagtatctgtttaggccagaggagatcgagctgctgatctgcggcagccgcaatctggactttcaggccctggaggagaccacagagtacgacggcggctatacccgggactccgtgctgatcagagagttctgggagatcgtgcactcttttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatga

SEQ ID NO: 11 - DNA, 22 bp, Artificial Sequence
  miRNA183 targeting sequence
agtgaattctaccagtgccata

SEQ ID NO: 12 - DNA, 466 bp, Artificial Sequence
  hSynapsin promoter
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcg

SEQ ID NO: 13 - DNA, 232 bp, Artificial Sequence
  SV40 polyA
cgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 14 - DNA, 2211 bp, adeno-associated virus hu68
  CDS (1)..(2211): AAVhu68 Capsid
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 15 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 2211 bp, adeno-associated virus hu68
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 17 - DNA, 2211 bp, adeno-associated virus rh.91
  CDS (1)..(2211): AAVrh.91 capsid
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 18 - protein, 736 aa, adeno-associated virus rh.91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 19 - DNA, 2211 bp, adeno-associated virus rh.91
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 20 - DNA, 24 bp, Artificial Sequence
  miRNA182 targeting sequence
agtgtgagttctaccattgccaaa

SEQ ID NO: 21 - protein, 872 aa, Artificial Sequence
  hUBE3a isoform 3 amino acid sequence
MATACKRSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 22 - DNA, 3398 bp, Artificial Sequence
  expression cassette hSyn.hUbe3a-1.GSco.4XmiRNA183.SV40
  promoter (1)..(466): hSynapsin
  misc_feature (478)..(483): Kozak
  misc_feature (484)..(3045): UBE3A-1
  misc_feature (3052)..(3073): miRNA183
  misc_feature (3078)..(3099): miRNA183
  misc_feature (3106)..(3127): miRNA183
  misc_feature (3134)..(3155): miRNA183
  polyA_signal (3167)..(3398): SV40 late polyA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatgaagagggcagcagcaaagcacctgatcgagagatactatcaccagctgaccgagggatgcggaaacgaggcatgtacaaacgagttctgcgcctcctgtcccacctttctgaggatggataacaacgccgccgccatcaaggccctggagctgtacaagatcaacgccaagctgtgcgacccccaccctagcaagaagggcgccagctccgcctatctggagaactccaagggcgcccctaacaatagctgttccgagatcaagatgaataagaagggcgcccggatcgatttcaaggacgtgacctacctgacagaggagaaggtgtacgagatcctggagctgtgccgggagagagaggattacagcccactgatcagagtgatcggcagagtgttctctagcgccgaggccctggtgcagtcctttagaaaggtgaagcagcacacaaaggaggagctgaagtctctgcaggccaaggacgaggacaaggacgaggacgagaaggagaaggcagcctgttctgccgcagcaatggaggaggacagcgaggcatcctctagccggatcggcgattcctctcaaggcgacaacaatctgcagaagctgggccccgacgacgtgagcgtggatatcgacgccatccggagagtgtacacaagactgctgagcaacgagaagatcgagaccgccttcctgaacgccctggtgtatctgagccctaatgtggagtgcgatctgacctaccacaacgtgtactcccgggacccaaactacctgaatctgttcatcatcgtgatggagaacagaaatctgcactcccccgagtatctggagatggccctgcctctgttttgtaaggccatgtccaagctgcctctggcagcacagggcaagctgatcaggctgtggtctaagtacaacgccgatcagatcaggcgcatgatggagaccttccagcagctgatcacatacaaagtgatctctaacgagtttaatagccgcaacctggtgaacgacgacgacgccatcgtggccgcctctaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtactccgagaggcgcatcaccgtgctgtattctctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtctaaggagttctttcagctggtggtggaggagatcttcaaccccgatatcggcatgtttacctacgacgagagcacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgactcccaccccgtgctgtaccagtctctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaatagaaaggagttcgtgaacctgtacagcgattatatcctgaataagtccgtggagaagcagttcaaggcctttaggcgcggcttccacatggtgaccaacgagagccctctgaagtatctgtttaggccagaggagatcgagctgctgatctgcggctcccgcaatctggactttcaggccctggaggagaccacagagtacgacggcggctataccagggactctgtgctgatccgcgagttctgggagatcgtgcacagctttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccagtgaattctaccagtgccataggatagtgaattctaccagtgccatacacgtgagtgaattctaccagtgccatagcatgcagtgaattctaccagtgccataggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 23 - DNA, 3294 bp, Artificial Sequence
  expression cassette hSyn.hUbe3a-1.GSco.SV40
  promoter (1)..(466): hSynapsin promoter
  misc_feature (478)..(483): Kozak
  misc_feature (484)..(3045): UBE3A-1
  polyA_signal (3063)..(3294): SV40 late polyA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatgaagagggcagcagcaaagcacctgatcgagagatactatcaccagctgaccgagggatgcggaaacgaggcatgtacaaacgagttctgcgcctcctgtcccacctttctgaggatggataacaacgccgccgccatcaaggccctggagctgtacaagatcaacgccaagctgtgcgacccccaccctagcaagaagggcgccagctccgcctatctggagaactccaagggcgcccctaacaatagctgttccgagatcaagatgaataagaagggcgcccggatcgatttcaaggacgtgacctacctgacagaggagaaggtgtacgagatcctggagctgtgccgggagagagaggattacagcccactgatcagagtgatcggcagagtgttctctagcgccgaggccctggtgcagtcctttagaaaggtgaagcagcacacaaaggaggagctgaagtctctgcaggccaaggacgaggacaaggacgaggacgagaaggagaaggcagcctgttctgccgcagcaatggaggaggacagcgaggcatcctctagccggatcggcgattcctctcaaggcgacaacaatctgcagaagctgggccccgacgacgtgagcgtggatatcgacgccatccggagagtgtacacaagactgctgagcaacgagaagatcgagaccgccttcctgaacgccctggtgtatctgagccctaatgtggagtgcgatctgacctaccacaacgtgtactcccgggacccaaactacctgaatctgttcatcatcgtgatggagaacagaaatctgcactcccccgagtatctggagatggccctgcctctgttttgtaaggccatgtccaagctgcctctggcagcacagggcaagctgatcaggctgtggtctaagtacaacgccgatcagatcaggcgcatgatggagaccttccagcagctgatcacatacaaagtgatctctaacgagtttaatagccgcaacctggtgaacgacgacgacgccatcgtggccgcctctaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtactccgagaggcgcatcaccgtgctgtattctctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtctaaggagttctttcagctggtggtggaggagatcttcaaccccgatatcggcatgtttacctacgacgagagcacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgactcccaccccgtgctgtaccagtctctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaatagaaaggagttcgtgaacctgtacagcgattatatcctgaataagtccgtggagaagcagttcaaggcctttaggcgcggcttccacatggtgaccaacgagagccctctgaagtatctgtttaggccagaggagatcgagctgctgatctgcggctcccgcaatctggactttcaggccctggaggagaccacagagtacgacggcggctataccagggactctgtgctgatccgcgagttctgggagatcgtgcacagctttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 24 - DNA, 3467 bp, Artificial Sequence
  expression cassette hSyn.hUbe3a-2.GSco.4XmiRNA183.SV40
  promoter (1)..(466): hSynapsin
  misc_feature (478)..(483): Kozak
  misc_feature (484)..(3114): hUBE3A-2
  misc_feature (3121)..(3142): miRNA183
  misc_feature (3147)..(3168): miRNA183
  misc_feature (3175)..(3196): miRNA183
  misc_feature (3203)..(3224): miRNA183
  polyA_signal (3236)..(3467): SV40 late polyA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggagaagctgcaccagtgctactggaagtctggcgagcctcagagcgacgatatcgaggcaagcaggatgaagagagcagcagccaagcacctgatcgagcggtactatcaccagctgaccgagggctgcggaaacgaggcctgtacaaacgagttctgcgcctcctgtccaacctttctgagaatggataacaacgccgccgccatcaaggccctggagctgtacaagatcaacgccaagctgtgcgacccccacccttctaagaagggcgccagctccgcctatctggagaacagcaagggcgcccccaacaatagctgttccgagatcaagatgaataagaagggcgccaggatcgatttcaaggacgtgacctacctgacagaggagaaggtgtacgagatcctggagctgtgccgggagagagaggattactcccctctgatcagagtgatcggacgcgtgttctctagcgccgaggccctggtgcagagctttcgcaaggtgaagcagcacacaaaggaggagctgaagtccctgcaggccaaggacgaggacaaggacgaggacgagaaggagaaggcagcttgttccgccgcagcaatggaggaggactctgaggcctcctctagcaggatcggcgattcctctcaaggcgacaacaatctgcagaagctgggccccgacgacgtgagcgtggatatcgacgccatccggagagtgtacacacgcctgctgtctaacgagaagatcgagaccgccttcctgaacgccctggtgtatctgtctcctaatgtggagtgcgatctgacctaccacaacgtgtacagccgggacccaaactacctgaatctgttcatcatcgtgatggagaacagaaatctgcactctcccgagtatctggagatggccctgcctctgttttgtaaggccatgagcaagctgccactggcagcacagggcaagctgatcaggctgtggtccaagtacaacgccgatcagatcaggcgcatgatggagaccttccagcagctgatcacatataaagtgatctccaacgagtttaattctagaaacctggtgaacgacgacgacgccatcgtggccgccagcaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtacagcgagaggcgcatcaccgtgctgtattccctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtccaaggagttctttcagctggtggtggaggagatcttcaaccccgatatcggcatgtttacctacgacgagtctacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgacagccaccccgtgctgtaccagtccctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaataggaaggagttcgtgaacctgtactctgattatatcctgaataagagcgtggagaagcagttcaaggcctttaggcgcggcttccacatggtgaccaacgagtctcctctgaagtatctgtttaggccagaggagatcgagctgctgatctgcggcagccgcaatctggactttcaggccctggaggagaccacagagtacgacggcggctatacccgggactccgtgctgatcagagagttctgggagatcgtgcactcttttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccagtgaattctaccagtgccataggatagtgaattctaccagtgccatacacgtgagtgaattctaccagtgccatagcatgcagtgaattctaccagtgccataggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 25 - DNA, 3363 bp, Artificial Sequence
  expression cassette hSyn.hUbe3a-2.GSco.SV40
  promoter (1)..(466): hSynapsin
  misc_feature (478)..(483): Kozak
  misc_feature (484)..(3114): hUBE3A-2
  polyA_signal (3132)..(3363): SV40 PolyA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggagaagctgcaccagtgctactggaagtctggcgagcctcagagcgacgatatcgaggcaagcaggatgaagagagcagcagccaagcacctgatcgagcggtactatcaccagctgaccgagggctgcggaaacgaggcctgtacaaacgagttctgcgcctcctgtccaacctttctgagaatggataacaacgccgccgccatcaaggccctggagctgtacaagatcaacgccaagctgtgcgacccccacccttctaagaagggcgccagctccgcctatctggagaacagcaagggcgcccccaacaatagctgttccgagatcaagatgaataagaagggcgccaggatcgatttcaaggacgtgacctacctgacagaggagaaggtgtacgagatcctggagctgtgccgggagagagaggattactcccctctgatcagagtgatcggacgcgtgttctctagcgccgaggccctggtgcagagctttcgcaaggtgaagcagcacacaaaggaggagctgaagtccctgcaggccaaggacgaggacaaggacgaggacgagaaggagaaggcagcttgttccgccgcagcaatggaggaggactctgaggcctcctctagcaggatcggcgattcctctcaaggcgacaacaatctgcagaagctgggccccgacgacgtgagcgtggatatcgacgccatccggagagtgtacacacgcctgctgtctaacgagaagatcgagaccgccttcctgaacgccctggtgtatctgtctcctaatgtggagtgcgatctgacctaccacaacgtgtacagccgggacccaaactacctgaatctgttcatcatcgtgatggagaacagaaatctgcactctcccgagtatctggagatggccctgcctctgttttgtaaggccatgagcaagctgccactggcagcacagggcaagctgatcaggctgtggtccaagtacaacgccgatcagatcaggcgcatgatggagaccttccagcagctgatcacatataaagtgatctccaacgagtttaattctagaaacctggtgaacgacgacgacgccatcgtggccgccagcaagtgcctgaagatggtgtactacgccaacgtggtgggcggcgaggtggacacaaaccacaacgaggaggacgacgaggagccaatccccgagagctccgagctgaccctgcaggagctgctgggagaggagcggagaaataagaagggaccaagggtggatcctctggagaccgagctgggcgtgaagacactggactgcagaaagcctctgatcccattcgaggagtttatcaacgagcccctgaacgaggtgctggagatggataaggactacaccttctttaaggtggagacagagaacaagttcagctttatgacctgtcctttcatcctgaacgccgtgaccaagaatctgggcctgtactacgataacaggatccgcatgtacagcgagaggcgcatcaccgtgctgtattccctggtgcagggccagcagctgaatccttacctgaggctgaaggtgcggagagaccacatcatcgatgacgccctggtgcgcctggagatgatcgccatggagaatccagccgatctgaagaagcagctgtacgtggagtttgagggagagcagggagtggacgagggaggcgtgtccaaggagttctttcagctggtggtggaggagatcttcaaccccgatatcggcatgtttacctacgacgagtctacaaagctgttctggtttaatccttcttccttcgagaccgagggccagtttacactgatcggcatcgtgctgggcctggccatctacaacaattgtatcctggacgtgcacttcccaatggtggtgtataggaagctgatgggcaagaagggcacctttcgcgatctgggcgacagccaccccgtgctgtaccagtccctgaaggatctgctggagtatgagggcaacgtggaggatgacatgatgatcaccttccagatctcccagacagacctgtttggcaacccaatgatgtacgatctgaaggagaacggcgacaagatccccatcacaaacgagaataggaaggagttcgtgaacctgtactctgattatatcctgaataagagcgtggagaagcagttcaaggcctttaggcgcggcttccacatggtgaccaacgagtctcctctgaagtatctgtttaggccagaggagatcgagctgctgatctgcggcagccgcaatctggactttcaggccctggaggagaccacagagtacgacggcggctatacccgggactccgtgctgatcagagagttctgggagatcgtgcactcttttacagacgagcagaagcggctgttcctgcagtttaccaccggcaccgacagagcaccagtgggaggactgggcaagctgaagatgatcatcgccaagaacggcccagacacagagaggctgcccaccagccacacctgtttcaacgtgctgctgctgcccgagtactcctctaaggagaagctgaaggagcgcctgctgaaggccatcacctacgccaagggctttggcatgctgtgatgaggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

(19) United States
(12) Patent Application Publication
(10) Pub. No.: US 2023/0414785 A1
(43) Pub. Date: Dec. 28, 2023

(54) COMPOSITIONS AND USES THEREOF FOR TREATMENT OF ANGELMAN SYNDROME

(71) Applicant: The Trustees of the University of Pennsylvania, Philadelphia, PA (US)

(72) Inventors: Justin Percival, Merion Station, PA (US); Ralf Schmid, Norfolk, MA (US); James M. Wilson, Philadelphia, PA (US)

(21) Appl. No.: 18/254,893

(22) PCT Filed: Dec. 1, 2021
(86) PCT No.: PCT/US2021/061346

§ 371 Date: May 30, 2023

Related U.S. Application Data
(60) Provisional application No. 63/119,860, filed on Dec. 1, 2020.
(60) Provisional application No. 63/179,807, filed on Apr. 26, 2021.

(51) Int. Cl. A61K 48/00; A61P 25/00

(52) U.S. Cl. CPC A61K 48/0058; A61K 48/0008; A61P 25/00

(57) ABSTRACT

A rAAV having a vector genome with a UBE3A coding sequence is provided. Also provided is a method for treating one or more symptoms of Angelman syndrome (AS) in a patient having deficient UBE3A expression in neurons, wherein the method comprises delivering a rAAV having a nucleic acid sequence which encodes UBE3A.

### BACKGROUND OF THE INVENTION

Angelman syndrome (AS) is a rare genetic disorder that affects 500,000 individuals worldwide. The primary symptoms of AS include intellectual disability, motor dysfunction, ataxia, absence of speech, severe seizures and distinctive behavioral features. Most individuals with AS exhibit a loss-of-function of the maternally inherited UBE3A allele, which encodes a HECT E3 ubiquitin ligase that links ubiquitin to substrates targeting them for degradation. 65-70% of AS cases result from class I mutations involving de novo deletion of the maternal chromosome 15q11-q13 [Angelman H., Puppet' children. A report on three cases, Dev Med Child Neurol, 1965, 7: 681-88; Mertz L G et al., Angelman syndrome in Denmark. Birth incidence, genetic findings, and age at diagnosis, Am J Med Genet A, 2013, 161A (9): 2197-2203, epub Aug. 2, 2013; Clayton-Smith J., and Laan L., Angelman syndrome: a review of the clinical and genetic types, J Med Genet, 2003, 40(2): 87-95, pub Feb. 1, 2003; Khatri N and Man H, The Autism and Angelman Syndrome Protein Ube3A/E6AP: The Gene, E3 Ligase Ubiquination Targets and Neurobiological Functions, Front Mol Neurosci, 2009, 12(109): 1-12, epub Apr. 30, 2019].

Three UBE3A isoforms have been described in mice, with isoform 2 and 3 having been identified as the major UBE3A splice variants in mice. (Valluy, J. et al., A coding-independent function of an alternative Ube3a transcript during neuronal development, Nat Neurosci, 2015, 18(5): 666-673, epub Apr. 13, 2015; Trezza R A et al., Loss of nuclear UBE3a causes electrophysiological and behavioral deficits in mice and is associated with Angelman syndrome, Nat Neurosci, 2019, 22(8): 1235-1247, epub Jun. 24, 2019; Zampeta F I et al., Conserved UBE3a subcellular distribution between human and mice is facilitated by non-homologous isoforms, Hum Mol Genet, 2020, 29(18):3032-3043, epub Sep. 2, 2020). Loss of hUBE3A isoform 1 occurs in individuals with “mild” Angelman Syndrome (Sadhwani, A et al., Two Angelman families with unusually advanced neurodevelopment carry a start codon variant in the most highly expressed UBE3a isoform, Am J Med Genet A, 2018, 176(7): 1641-1647, epub May 7, 2018) that still express nuclear hUBE3A isoform 3 and cytoplasmic hUBE3A isoform 2.

At present, there is no cure for AS. Current treatments are palliative and focus on the management of medical and developmental issues, including seizures. The absence of treatment options underscores the critical unmet need for novel therapies for AS.

### SUMMARY OF THE INVENTION

A novel adeno-associated virus (AAV) based gene replacement therapy based on UBE3A-isoform 1 which is useful and well-tolerated is provided herein. The compositions and methods can mitigate motor and behavior deficits associated with Angelman Syndrome (AS), as assessed in animal models of Angelman. In one embodiment, a composition comprises a stock of recombinant adeno-associated virus (rAAV) useful for treatment of AS, the rAAV comprising an AAV capsid and a vector genome packaged therein, said vector genome comprising: (a) an AAV 5′ inverted terminal repeat (ITR); (b) a UBE3A nucleic acid sequence comprising SEQ ID NO: 9 or a sequence 95% identical thereto encoding UBE3A isoform 1 protein (SEQ ID NO: 2), wherein the nucleic acid is operably linked to regulatory elements which regulate expression of the UBE3A protein in human cells; (c) regulatory elements which direct expression of the UBE3A of (b); and (d) an AAV 3′ ITR. In certain embodiments, the regulatory elements comprise a neuron-specific promoter. In certain embodiments, the neuron-specific promoter is a synapsin promoter. In certain embodiments, the synapsin promoter is a shortened promoter having the nucleic acid sequence of SEQ ID NO: 12. In certain embodiments, the regulatory elements comprise a constitutive promoter. In certain embodiments, the regulatory elements further comprise one or more enhancer and one or more introns. In certain embodiments, the regulatory sequences further comprise one or more targeting sequences for miR182 (SEQ ID NO: 20) and/or miR183 (SEQ ID NO: 11), said targeting sequences operably linked to the UBE3A nucleic acid sequence. In certain embodiments, the regulatory sequences further comprise one or more targeting sequences for miR selected from miR182 and/or miR183, said targeting sequences located downstream of the UBE3A nucleic acid sequence. In certain embodiments, the regulatory sequences further comprise four targeting sequences for miR183, said targeting sequences located downstream of the UBE3A nucleic acid sequence. In certain embodiments, the regulatory sequences comprise four copies of SEQ ID NO: 11. In certain embodiments, the AAV capsid is a AAVhu68 capsid. In certain embodiments, the AAV capsid is a AAVhu68 capsid generated from expression of the nucleic acid sequence of SEQ ID NO: 14 or SEQ ID NO: 16. In certain embodiments, the AAV capsid is a AAVrh91 capsid. In certain embodiments, the AAV capsid is a AAVrh91 capsid expressed from the nucleic acid sequence of SEQ ID NO: 17 or SEQ ID NO: 19. In certain embodiments, the composition is an aqueous suspension further comprising a physiologically compatible carrier, buffer, adjuvant, and/or diluent.

In certain embodiments, a composition as described herein is useful for use in treating a patient having Angelman Syndrome. In certain embodiments, a composition as provided herein is useful in treating one or more symptoms of an Angelman Syndrome, optionally where the symptoms are selected from one or more of: delayed development, intellectual disability, severe speech impairment, ataxia and/or epilepsy. In certain embodiments, a method is provided for treating one or more symptoms of Angelman syndrome (AS) in a patient having deficient UBE3A expression in neurons via delivery of an expression cassette provided herein. In certain embodiments, the expression cassette treats symptoms selected from one or more of: delayed development, intellectual disability, severe speech impairment, ataxia and/or epilepsy. In certain embodiments, compositions are provided for intrathecal delivery to a patient. In certain embodiments, the patient is injected with at least 1×10¹⁰ to 1×10¹³ GC/kg of the rAAV carrying the engineered UBE3A coding sequence.

In certain embodiments, the method provides for improvement of a symptom of Angelman disease, including one or more of delayed development, intellectual disability, severe speech impairment, ataxia and/or epilepsy.

Other aspects and advantages of these methods and compositions are described further in the following detailed description.

### BRIEF DESCRIPTION OF THE FIGURES

FIGS. 1A to 1C provide schematic illustrations of expression cassettes and vector genomes without miR sequences for modulating dorsal root ganglia expression and toxicity. FIG. 1A provides a schematic illustration of a vector genome in which the 5′ AAV inverted terminal repeat (ITR) and the 3′ AAV ITR flank the expression cassette for hUBE3A-isoform 1. The expression cassette contains an engineered hUBE3A-isoform 1 coding sequence (encoding 852 amino acids; SEQ ID NO: 2) under the control a modified synapsin promoter. The hUBE3A coding sequence includes an amino-terminal zinc-finger of Ube3A ligase (AZUL), the HECT domain and RCC1-like domain 2 (HERC2), the E6 protein binding domain (E6BD), and a region homologous to E6-AP carboxyl terminus (HECT), and an SV40 polyA. FIG. 1B provides a schemical illustration of a vector genome in which the AAV ITRs flank an expression cassette for hUBE3A-isoform 2. The encoded isoform 2 protein is 875 amino acids in length (SEQ ID NO: 6). FIG. 1C provides a schemical illustration of a vector genome in which the AAV ITRs flank an expression cassette for hUBE3A-isoform 3. The encoded isoform 3 protein is 872 amino acids in length (SEQ ID NO: 21).

FIGS. 2A and 2B illustrate results of an evaluation of vector biodistribution and mRNA expression of rAAVhu68.UBE3A-isoform 1 with 4 copies of miR183 (4×miR183) target sequences (light circles) or without 4×miR183 sequences (dark sequences) in non-human primates (NHPs). FIG. 2A illustrates UBE3A isoform 1 vector biodistribution in NHPs as measured in genome copies (GC)/diploid genome in cerebellum, caudate nucleus, hippocampus, frontal cortex, occipital cortex, medulla, parietal cortex, temporal cortex, thalamus, DRG cervical, DRG thoracic, DRG lumbar, spinal cord cervical, spinal cord thoracic, spinal cord lumbar. FIG. 2B illustrates UBE3A isoform 1 mRNA expression after treatment with rAAVhu68.UBE3A-isoform 1 with 4 copies of miR183 (4×miR183) target sequences (light circles) or without 4×miR183 sequences (dark sequences) in spinal cord and DRGs of NHPs. FIG. 2C illustrates UBE3A isoform 1 in cerebellum, caudate nucleus, hippocampus, frontal cortex, occipital cortex medulla, parietal cortex, temporal cortex, thalamus, DRG cervical, DRG thoracic, DRG lumbar, spinal cord cervical, spinal cord thoracic, spinal cord lumbar, cerebrum (negative control).

FIGS. 3A and 3B show that AAVhu68-UBE3A-isoforml 4×miR183 vectors do not cause significant dorsal root ganglia (DRG) toxicity in (3-4 years old) rhesus macaques. hUBE3A-1 (Group 1) or hUbe3a-1-4×miR183 (Group 2) vectors do not cause significant AAV-induced dorsal root ganglion toxicity in rhesus macaques 35 days post cisterna magna (ICM) administration at a dose of 3×10¹³ GC/animal. DRG-associated toxicity was only observed in the spinal cord and peripheral nerves of Group 1 animal 192285. The DRG in animals across both groups were unremarkable (FIG. 3A) with only sporadic minimal mononuclear cell infiltrates (FIG. 3B, circle) and no evidence of neuronal degeneration (3/3 animals, Group 1; 3/3 animals, Group 2).

FIGS. 4A and 4B show the impact of rAAVhu68.synapsin-UBE3A isoform 1 in peripheral nerve in (3-4 years old) rhesus macaques. Impact of minimal focal axonopathy in hind limb nerves (arrowhead). FIG. 4A shows Animal AAVhu68.synapsin-UBE3A isoform 1 in peripheral nerves. FIG. 4B shows Animals 192275 and 192297 exhibited 192285 exhibited mild multifocal axonopathy (arrowheads) and mononuclear cell infiltrates (oval) in the forelimb right median nerve.

FIG. 5 shows quantification of UBE3A protein positive neurons in treated UBE3A^{m-/p+} neonatal mice. Treatment comprised of administration of AAV-PHP.B-hSyn-UBE3A-isoform 1 at a dose of 1×10¹¹ GC/animal via intracerebroventricular (ICV). In cortex, hippocampus, thalamus, hypothalamus and midbrain, 42-68% of neurons expressed UBE3A protein (normalized to UBE3A positive neurons in the respective WT tissue).

FIG. 6 shows quantification of UBE3A protein positive neurons in treated UBE3A^{m-/p+} neonatal mice. Treatment comprised administration of AAV-PHP.B-hSyn-UBE3A-isoform 1 at a dose of 1×10¹⁰ GC/animal via intracerebroventricular (ICV). In cortex, hippocampus, thalamus, hypothalamus and midbrain, 20-50% of neurons expressed UBE3A protein (normalized to UBE3A positive neurons in the respective WT tissue).

FIGS. 7A to 7I show fluorescent images of engineered human UBE3A isoform 1 (hUBE3A-1) transcript localization in dorsal root ganglia (cervical, thoracic and dorsal segments) from three treated non-human primates (NHP-1, -2, -3; in a 35-day study). Treatment comprised administration of AAV-PHP.B-hSyn-UBE3A-isoform 1 at a dose of 3×10¹³ GC/animal via cisterna magna (ICM) route. Images of regions of interest were taken at various magnifications, and images are presented at 20× magnification. FIG. 7A shows fluorescent image of engineered hUBE3A-1 transcript localization in cervical segment of dorsal root ganglia from NHP-1. FIG. 7B shows fluorescent image of engineered hUBE3A-1 transcript localization in cervical segment of dorsal root ganglia from NHP-2. FIG. 7C shows fluorescent image of engineered hUBE3A-1 transcript localization in cervical segment of dorsal root ganglia from NHP-3. FIG. 7D shows fluorescent image of engineered hUBE3A-1 transcript localization in thoracic segment of dorsal root ganglia from NHP-1. FIG. 7E shows fluorescent image of engineered hUBE3A-1 transcript localization in thoracic segment of dorsal root ganglia from NHP-2. FIG. 7F shows fluorescent image of engineered hUBE3A-1 transcript localization in thoracic segment of dorsal root ganglia from NHP-3. FIG. 7G shows fluorescent image of engineered hUBE3A-1 transcript localization in lumbar segment of dorsal root ganglia from NHP-1. FIG. 7H shows fluorescent image of engineered hUBE3A-1 transcript localization in lumbar segment of dorsal root ganglia from NHP-2. FIG. 7I shows fluorescent image of engineered hUBE3A-1 transcript localization in lumbar segment of dorsal root ganglia from NHP-3.

FIG. 8 shows expression of engineered UBE3A isoform 1 in brain of UBE3A^{m-/p+} and wild type mice following ICV injection with AAV-PHP.B-hSyn-hUBE3A-isol.

FIGS. 9A and 9B show results of motor coordination behavioral test performed at 8-10 weeks of age after neonatal wild type or AS (UBE3A^{m-/p+}) mice were injected intracerebroventricularly (ICV) with either AAV-PHP.B-synapsin-UBE3A-isoform 1 or isoform 2 vectors at a dose of 1×10¹¹ genome copies (GC) per animal. FIG. 9A shows motor coordination in WT and AS mice following treatment with AAV-PHP.B-synapsin-UBE3A-isoform 1. FIG. 9B shows motor coordination in WT and AS mice following treatment with AAV-PHP.B-synapsin-UBE3A-isoform 2.

FIGS. 10A to 10D show results of the nest building ability behavioral test performed at 8-10 weeks of age after neonatal wild type or AS (UBE3A^{m-/p+}) mice were injected intracerebroventricularly (ICV) with either AAV-PHP.B-synapsin-UBE3A-isoform 1 or isoform 2 vectors at a dose of 1×10¹¹ genome copies (GC) per animal. FIG. 10 A shows nest building score in WT and AS mice following treatment with AAV-PHP.B-synapsin-UBE3A-isoform 1. FIG. 10B shows percentage of unused nestlet by WT and AS mice following treatment with AAV-PHP.B-synapsin-UBE3A-isoform 1. FIG. 10 C shows nest building score in WT and AS mice following treatment with AAV-PHP.B-synapsin-UBE3A-isoform 2. FIG. 10D shows percentage of unused nestlet by WT and AS mice following treatment with AAV-PHP.B-synapsin-UBE3A-isoform 2.

FIGS. 11A to 11D show results of the catwalk (stride length and gait improvement) behavioral test performed at 8-10 weeks of age after neonatal wild type or AS (UBE3A^{m-/p+}) mice were injected intracerebroventricularly (ICV) with AAV-PHP.B-synapsin-UBE3A-isoform 1 vector at a dose of 1×10¹¹ genome copies (GC) per animal. FIG. 11 A shows stride length of right hind (RH) limb in WT and AS mice following treatment with AAV-PHP.B-synapsin-UBE3A-isoform 1. FIG. 11B shows stride length of left hind (LH) limb in WT and AS mice following treatment with AAV-PHP.B-synapsin-UBE3A-isoform 1. FIG. 11 C shows stride length of right hind (RH) limb in WT and AS mice following treatment with AAV-PHP.B-synapsin-UBE3A-isoform 1. FIG. 11D shows stride length of left hind (LH) limb in WT and AS mice following treatment with AAV-PHP.B-synapsin-UBE3A-isoform 1.

FIGS. 12A to 2C show results of toxicity study in dorsal root ganglia (drg) in NHPs after 35 days post treatment with AAV-hu68-hSyn-UBE3A-isoform 1 at a dose of 3×10¹³ GC/animal via cisterna magna (ICM) route (plotted as pathology grade scored 0-5). FIG. 12A shows scored pathology grade in cervical segment of DRG of NHPs. FIG. 12B shows scored pathology grade in thoracic segment of DRG of NHPs. FIG. 12C shows scored pathology grade in lumbar segment of DRG of NHPs.

FIGS. 13A to 13C show results of toxicity study in spinal cord in NHPs after 35 days post treatment with AAV-hu68-hSyn-UBE3A-isoform 1 at a dose of 3×10¹³ GC/animal via cisterna magna (ICM) route (plotted as pathology grade scored 0-5). FIG. 13A shows scored pathology grade in cervical segment of spinal cord of NHPs. FIG. 13B shows scored pathology grade in thoracic segment of spinal cord of NHPs. FIG. 13C shows scored pathology grade in lumbar segment of spinal cord of NHPs.

FIG. 14 shows results of toxicity study in peripheral nerve in NHPs after 35 days post treatment with AAV-hu68-hSyn-UBE3A-isoform 1 at a dose of 3×10¹³ GC/animal via cisterna magna (ICM) route (plotted as axonopathy pathology grade scored 0-5).

FIGS. 15A to 15C show results of peripheral nerve conduction study after 14- and 35-days post treatment with AAV-hu68-hSyn-UBE3A-isoform 1 at a dose of 3×10¹³ GC/animal via cisterna magna (ICM) route. FIG. 15A shows velocity measured as m/sec of left median nerve. FIG. 15 B shows results of peripheral nerve conduction study after 14- and 35-days post treatment with AAV-hu68-hSyn-UBE3A-isoform 1, measured peak-to-peak (PP) amplitude in mV. FIG. 15C shows results of peripheral nerve conduction study after 14- and 35-days post treatment with AAV-hu68-hSyn-UBE3A-isoform 1, measured negative peak (NP) amplitude in mV.

FIGS. 16A and 16B show quantification of UBE3A isoform 1 or isoform 2 protein positive neurons in treated UBE3A^{m-/p+} neonatal mice, plotted as percent positive neurons in cortex, hippocampus, thalamus, hypothalamus and midbrain, normalized with respect to UBE3A positive neurons in the WT tissue. Treatment comprised administration of AAV-PHP.B-hSyn-UBE3A-isoform 1 or isoform 2 at a dose of 1×10¹¹ GC/animal via intracerebroventricular (ICV). FIG. 16A shows percent of UBE3A isoform 1 positive neurons in cortex, hippocampus, thalamus, hypothalamus and midbrain in mice post treatment. FIG. 16B shows percent of UBE3A isoform 2 positive neurons in cortex, hippocampus, thalamus, hypothalamus and midbrain in mice post treatment.

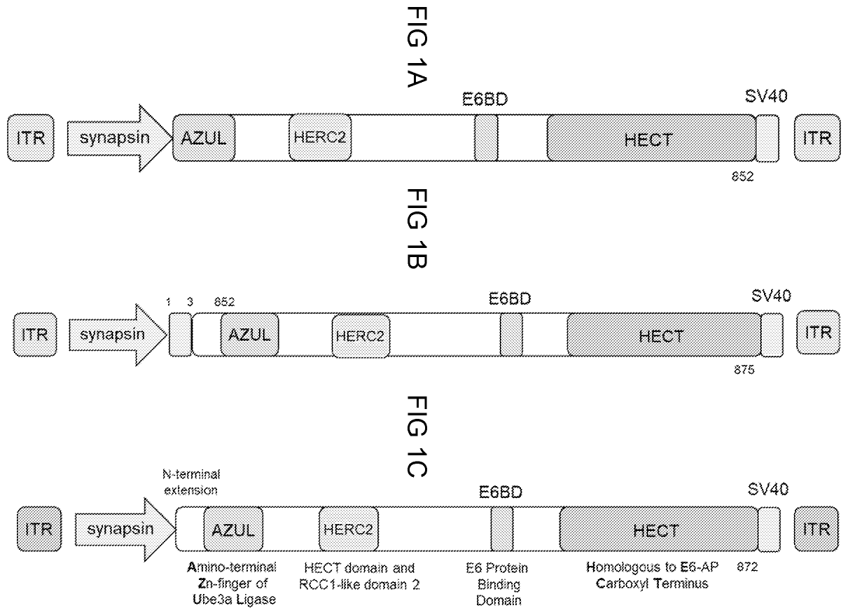

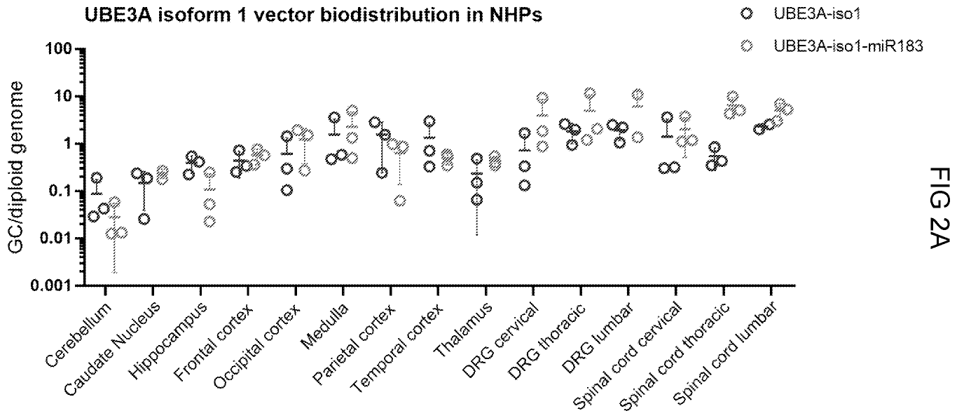

### DETAILED DESCRIPTION OF THE INVENTION

Provided herein are expression cassettes containing engineered UBE3A-isoform 1 coding sequences which when delivered (e.g., via rAAV-mediated gene replacement therapy) express UB3A isoform 1 at levels that treat symptoms of Angelman Syndrome. In certain embodiments, the regulatory elements in the expression cassette comprise up to eight, e.g., four to eight miR183 sequences to modulate dorsal root ganglion (DRG) expression and/or toxicity. In certain embodiments, these DRG-detargeting sequences are selected for use when expression at high levels and/or when systemic delivery is intended. In other embodiments, these sequences are included when intrathecal delivery is utilized.

In one embodiment, an expression cassette comprises an engineered UBE3A coding (nucleic acid sequence) operably linked to regulatory elements which regulate expression of the UBE3A protein in targeted human cells. In certain embodiments, the UBE3A coding sequence encodes UBE3A isoform 1 protein, which is reproduced in SEQ ID NO: 2. The hUBE3A isoform 1 protein includes several domains, including an amino-terminal zinc-finger of Ube3A ligase (AZUL), the HECT domain and RCC1-like domain 2 (HERC2), the E6 protein binding domain (E6BD), and a region homologous to E6-AP carboxyl terminus (HECT). Suitably, the engineered UBE3A isoform 1 coding sequences are the nucleic acid sequence of SEQ ID NO: 9 or a sequence at least 95% identical thereto encoding UBE3A isoform 1 protein (SEQ ID NO: 2). In certain embodiments, the sequence is 100% identical to the full-length of SEQ ID NO: 9. In other embodiments, the sequence is at least 95% identical, at least 97% identical, at least 98% identical, at least 99% identical, 99.5% identical to SEQ ID NO: 9. In certain embodiments, the UBE3A isoform 1 coding sequence is truncated at the 5′ or 3′ end, resulting in a truncation of the carboxy or N-terminus of the UBE3A isoform 1 protein.

In certain embodiments, the UBE3A coding sequences encoding UBE3A isoform 2 protein, which is reproduced in SEQ ID NO: 6. The hUBE3A isoform 2 protein includes several domains, including an amino-terminal zinc-finger of Ube3A ligase (AZUL), the HECT domain and RCC1-like domain 2 (HERC2), the E6 protein binding domain (E6BD), and a region homologous to E6-AP carboxyl terminus (HECT). Suitably, the engineered UBE3A isoform 2 coding sequences are the nucleic acid sequence of SEQ ID NO: 10 or a sequence at least 95% identical thereto encoding UBE3A isoform 2 protein (SEQ ID NO: 6). In certain embodiments, the sequence is 100% identical to the full-length of SEQ ID NO: 10. In other embodiments, the sequence is at least 95% identical, at least 97% identical, at least 98% identical, at least 99% identical, 99.5% identical to SEQ ID NO: 10. In certain embodiments, the UBE3A isoform 2 coding sequence is truncated at the 5′ or 3′ end, resulting in a truncation of the carboxy or N-terminus of the UBE3A isoform 2 protein. In certain embodiments, the UBE3a coding sequences encoding UBE3A isoform 3 protein, which is reproduced in SEQ ID NO: 21 (UNIPROT ID No: Q05086-3).

In certain embodiments, the expression cassette comprises UBE3A coding sequence, wherein optionally the UBE3A coding sequence encodes a fusion protein comprising a signal peptide and a functional UBE3A protein. In certain embodiments, the expression cassette comprises UBE3A coding sequence, wherein optionally the UBE3A coding sequence encodes for a fusion protein comprising an uptake peptide fused to a functional UBE3A protein. In certain embodiments, the expression cassette UBE3A coding sequence, wherein optionally the UBE3A coding sequence encodes a fusion protein comprising the UBE3A protein fused to a signal peptide and/or an uptake peptide. In some embodiments, the expression cassette comprises UBE3A coding sequence, wherein optionally the signal peptide and/or the uptake peptide are located at either 5′ or 3′ of the UBE3A coding sequence to afford a fusion protein comprising the signal peptide and/or uptake peptide at the N-terminus of the UBE3A protein, a fusion protein comprising the signal peptide and/or uptake peptide at the C-terminus of the UBE3A protein, or a fusion protein comprising a signal peptide at the N-terminus of the UBE3A protein or a fusion protein with a signal peptide or uptake peptide at the C-terminus of the UBE3A protein, or combinations thereof. In certain embodiments, the signal peptide is a binding immunoglobulin protein (BiP) signal peptide. In certain embodiments, the signal peptide is a Gaussia signal peptide. See also, U.S. Pat. No. 9,279,007 B2 ((corresponding International Patent Application No. WO2012/071422; binding immunoglobulin protein (BiP) signal peptide), U.S. Pat. No. 10,874,750 B2 (corresponding International Patent Application No. WO2019/213180A1; binding immunoglobulin protein (BiP) signal peptide and a Gaussia signal peptide) which are incorporated herein by reference in its entirety. In certain embodiments, optionally the UBE3A is a fusion protein comprising peptide which enhances expression, secretion, and cellular uptake. In certain embodiments, optionally the UBE3A is a fusion protein comprising peptide which is a cystatin peptide sequence. See also, U.S. Pat. No. 9,567,369 which is incorporated herein by reference in its entirety. In certain embodiments, optionally the UBE3A is a fusion protein comprising peptide which is derivative from HIV TATk peptide (e.g., TATk28, TATk11). See also, WO2015/128746A2 (U.S. Pat. No. 10,907,138 B2), WO2018/005617A2 (US 2021/0268072), WO2019/108924A2, WO2020/250081A1 and WO2021/087282A1, which are incorporated herein by reference in its entirety. In certain embodiments, the peptide is IGF2 peptide. See also, WO2021/072372 which is incorporated by reference in its entirety. In certain embodiments, optionally the UBE3A is a fusion protein comprising a peptide comprising a cell uptake sequence selected from penetratin, R6W3, HIV TAT, HIV TATk and pVEC. In certain embodiments, optionally the UBE3A is a fusion protein comprising a peptide comprising secretion sequence selected from insulin, GDNF, and IgK. See also, WO 2019/006107, which is incorporated herein by reference in its entirety.

In certain embodiments, the UBE3A isoform 1 expression cassette is selected for delivery as gene replacement therapy alone. In certain embodiments, the UBE3A isoform 1 expression cassette is selected for delivery as a gene replacement therapy in a regimen involving one or more other active components (e.g., short-term or long-term enzyme replacement therapy and/or substrate depletion therapy).

In one embodiment, a composition comprises a vector comprising an expression cassette for UBE3A isoform 1. Suitable vectors and vector genomes are described herein.

In other embodiments, a stock of recombination parvovirus vectors (e.g., recombinant adeno-associated virus) is provided. The rAAV comprise an AAV capsid and a vector genome packaged therein, said vector genome comprising: (a) an AAV 5′ inverted terminal repeat (ITR); (b) a UBE3A nucleic acid sequence comprising SEQ ID NO: 9 or a sequence 95% identical thereto encoding UBE3A isoform 1 protein (SEQ ID NO: 2), wherein the nucleic acid is operably linked to regulatory elements which regulate expression of the UBE3A protein in human cells; (c) regulatory elements which direct expression of the UBE3A of (b); and (d) an AAV 3′ ITR. Desirable AAV capsids include AAVhu68 and AAVrh91, which target desired cells in the central nervous system (CNS).

In certain embodiments, the rAAV comprises an AAV capsid and a vector genome packaged therein, wherein said vector genome comprises: (a) an AAV 5′ inverted terminal repeat (ITR); (b) optionally a peptide (e.g., signal or an uptake peptide); (c) a UBE3A nucleic acid sequence comprising SEQ ID NO: 9 or a sequence 95% identical thereto encoding UBE3A isoform 1 protein (SEQ ID NO: 2), wherein the nucleic acid sequence is operably linked to regulatory elements which regulate expression of the UBE3A protein in human cells; (d) optionally a peptide (regulatory and/or uptake peptide); (e) regulatory elements which direct expression of the UBE3A of (b); and (f) an AAV 3′ ITR. In certain embodiments, the signal peptide is BiP signal peptide. See also, U.S. Pat. No. 9,279,007 B2 ((corresponding International Patent Application No. WO2012/071422) and U.S. Pat. No. 10,874,750 B2 (corresponding International Patent Application No. WO2019/213180A1), which are incorporated herein by reference in its entirety. In certain embodiments, the regulatory peptide is IGF peptide. See also, WO2021/072372 which is incorporated by reference in its entirety. In certain embodiments, the signal peptide is a secretion signal peptide comprising secretion sequence selected from insulin, GDNF, and IgK. In certain embodiments, the uptake peptide comprising a cell uptake sequence selected from penetratin, R6W3, HIV TAT, HIV TATk and pVEC. See also, WO 2019/006107, which is incorporated herein by reference in its entirety.

In certain embodiments, the UBE3A is optionally a fusion protein comprising a signal peptide and/or an uptake peptide, as described herein. In certain embodiments, a signal peptide and/or an uptake peptide is located at either the amino (N)-terminus or at carboxy (C)-terminus. In certain embodiments, a signal peptide is located at the amino (N)-terminus. In certain embodiment, an uptake peptide is located at either N-terminus or C-terminus.

As used herein, a “stock” of rAAV refers to a population of rAAV. Despite heterogeneity in their capsid proteins due to deamidation, rAAV in a stock are expected to 5 share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

As used herein, “disease”, “disorder”, and “condition” are used interchangeably, to indicate an abnormal state in a subject. In one embodiment, the disease is Angelman syndrome (AS).

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an”, refers to one or more, for example, “a neuron”, is understood to represent one or more neuron(s). As such, the terms “a” (or “an”), “one or more,” and “at least one” is used interchangeably herein.

As used herein, the term “about” means a variability of plus or minus 10% from the reference given, unless otherwise specified.

In certain instances, the term “E+#” is used to reference an exponent. For example, 5E10 is 5×10¹⁰. These terms may be used interchangeably.

Nucleic acid sequences described herein can be cloned using routine molecular biology techniques, or generated de novo by DNA synthesis. The nucleic acid sequences encoding aspects of a UBE3A gene described herein are assembled and placed into any suitable genetic element, e.g., naked DNA, phage, transposon, cosmid, episome, etc., which transfers the sequences carried thereon to a host cell, e.g., for generating non-viral delivery systems (e.g., RNA-based systems, naked DNA, or the like), or for generating viral vectors in a packaging host cell, and/or for delivery to a host cells in a subject. In one embodiment, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

### Expression Cassettes

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

In some embodiments, the nucleic acid molecule which comprises a coding sequence is UBE3A coding sequence, and further comprises a promoter, and may include other regulatory sequences therefor. In certain embodiments, the expression cassette is used for generating a viral vector (e.g., a viral particle) which contains the coding sequence for the UBE3A described herein flanked by packaging signals of the viral genome and other expression control sequences such as those described herein. In some embodiments, the viral vector is an AAV viral vector, wherein the packaging signals are a 5′ AAV inverted terminal repeat (ITR) and a 3′ AAV ITR. Optionally, an expression cassette (and a vector genome) may comprise one or more dorsal root ganglion (drg)-miRNA targeting sequences in the UTR, e.g., to reduce drg toxicity and/or axonopathy. See, e.g., PCT/US2019/67872, filed Dec. 20, 2019 and now published as WO 2020/132455, U.S. Provisional Patent Application No. 63/023,593, filed May 12, 2020, and U.S. Provisional Patent Application No. 63/038,488, filed Jun. 12, 2020, all entitled “Compositions for Drg-Specific Reduction of Transgene Expression”, which are incorporated herein in their entireties.

As used herein, the term “operably linked” or “operatively associated” refers to both expression control sequences or regulatory elements that are contiguous with the gene of interest and expression control sequences that act in trans or at a distance to control the gene of interest.

As described herein, regulatory elements comprise but not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence).

In one embodiment, the expression cassette comprises regulatory elements which direct expression of a sequence encoding one or more elements of a gene replacement system for delivering UBE3A. In one embodiment, the regulatory elements comprise one or more promoters. In certain embodiments, the expression cassette includes a constitutive or a regulatable promoter. In certain embodiments, the promoter is a tissue-specific (e.g., neuron specific) promoter. In certain embodiments, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kugler S et al, Human synapsin 1 gene promoter confers highly neuron-specific long-term transgene expression from an adenoviral vector in the adult rat brain depending on the transduced area. Gene Ther. 2003 February; 10(4):337-47), a shorted synapsin promoter, such as provided in the Examples herein (see, e.g., SEQ ID NO: 12 for coding sequences), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5). Other suitable promoters include CAG promoter, which comprises (C) the cytomegalovirus (CMV) early enhancer element, (A) the promoter, the first exon and the first intron of chicken beta-actin gene, and (G) the splice acceptor of the rabbit beta-globin gene. See, e.g., Alexopoulou, Annika N., et al. BMC cell biology 9.1 (2008): 2. Although less desired, other promoters, such as viral promoters, constitutive promoters, inducible promoters, regulatable promoters (see, e.g., WO 2011/126808 and WO 2013/04943), or a promoter responsive to physiologic cues may be used may be utilized in the vectors described herein. In certain embodiments, the expression cassette includes an U6 promoter. In another embodiment, the regulatory elements comprise an enhancer. In a further embodiment, the enhancer(s) is selected from one or more of an APB enhancer, an ABPS enhancer, an alpha mic/bik enhancer, a TTR enhancer, an en34 enhancer, an ApoE enhancer, a CMV enhancer, or an RSV enhancer. In yet another embodiment, the regulatory elements comprise an intron. In a further embodiment, the intron is selected from CBA, human beta globin, IVS2, SV40, bGH, alpha-globulin, beta-globulin, collagen, ovalbumin, or p53. In one embodiment, the regulatory elements comprise a polyA. In a further embodiment, the polyA is a synthetic polyA or from bovine growth hormone (bGH), human growth hormone (hGH), SV40, such as provided in the Examples herein (see, e.g., SEQ ID NO: 13 for coding sequence), rabbit p-globin (RGB), or modified RGB (mRGB). In another embodiment, the regulatory elements may comprise a WPRE sequence. In yet another embodiment, the regulatory elements comprise a Kozak sequence.

In certain embodiments, the expression cassette comprises nucleic acid sequence of SEQ ID NO: 22 or a sequence at least about 90% identical thereto, which encodes for UBE3A comprising amino acid sequence of SEQ ID NO: 2. In certain embodiments, the expression cassette comprises nucleic acid sequence of SEQ ID NO: 23 or a sequence at least about 90% identical thereto, which encodes for UBE3A comprising amino acid sequence of SEQ ID NO: 4. In certain embodiments, the expression cassette comprises nucleic acid sequence of SEQ ID NO: 24 or a sequence at least about 90% identical thereto, which encodes for UBE3A comprising amino acid sequence of SEQ ID NO: 6. In certain embodiments, the expression cassette comprises nucleic acid sequence of SEQ ID NO: 25 or a sequence at least about 90% identical thereto, which encodes for UBE3A comprising amino acid sequence of SEQ ID NO: 8.

The term “expression” is used herein in its broadest meaning and comprises the production of RNA, of protein, or of both RNA and protein. With respect to RNA, the term “expression” or “translation” relates in particular to the production of peptides or proteins. Expression may be transient or may be stable.

Expression cassettes can be delivered via any suitable delivery system. Suitable non-viral delivery systems are known in the art (see, e.g., Ramamoorth and Narvekar. J Clin Diagn Res. 2015 January; 9(1):GE01-GE06, which is incorporated herein by reference) and can be readily selected by one of skill in the art and may include, e.g., naked DNA, naked RNA, dendrimers, PLGA, polymethacrylate, an inorganic particle, a lipid particle (e.g., a lipid nanoparticle or LNP), or a chitosan-based formulation.

In one embodiment, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

Provided herein are compositions comprising a nucleic acid sequence encoding one or more elements of a gene replacement system and methods of use thereof for replacing functional UBE3A.

Optionally, the expression cassette may include miRNA target sequences in the untranslated region(s). The miRNA target sequences are designed to be specifically recognized by miRNA present in cells in which transgene expression is undesirable and/or reduced levels of transgene expression are desired. In certain embodiments, the miRNA target sequences are located in the 3′ UTR, 5′ UTR, and/or in both 3′ and 5′ UTR. In some embodiments, the miRNA target sequences are operably linked to the regulatory sequences in the expression cassette. In certain embodiments, the expression cassette comprises at least two tandem repeats of DRG-specific miRNA target sequences, wherein the at least two tandem repeats comprise at least a first miRNA target sequence and at least a second miRNA target sequence which may be the same or different. In certain embodiments, the tandem miRNA target sequences are continuous or are separated by a spacer of 1 to 10 nucleic acids, wherein said spacer is not a miRNA target sequence.

In certain embodiments, the vector genome or expression cassette contains at least one miRNA target sequence that is a miR-183 (or miRNA183) target sequence. In certain embodiments, the vector genome or expression cassette contains a miR-183 target sequence that includes AGTGAATTCTACCAGTGCCATA (SEQ ID NO: 11), where the sequence complementary to the miR-183 seed sequence is underlined. In certain embodiments, the vector genome or expression cassette contains more than one copy (e.g., two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 11 and, thus, when aligned to SEQ ID NO: 11, there are one or more mismatches. In certain embodiments, a miR-183 target sequence comprises a sequence having at least 1, 2, 3, 4, 5, 6, 7, 8, 9, or 10 mismatches when aligned to SEQ ID NO: 11, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 11, i.e., a sequence that lacks at least 1, 2, 3, 4, 5, 6, 7, 8, 9, or 10 nucleotides at either or both the 5′ or 3′ ends of SEQ ID NO: 11. In certain embodiments, the expression cassette or vector genome comprises a transgene and one miR-183 target sequence. In yet other embodiments, the expression cassette or vector genome comprises at least two, three or four miR-183 target sequences.

In certain embodiments, the vector genome or expression cassette contains at least one miRNA target sequence that is a miR-182 target sequence. In certain embodiments, the vector genome or expression cassette contains a miR-182 target sequence that includes AGTGTGAGTTCTACCATTGCCAAA (SEQ ID NO: 20). In certain embodiments, the vector genome or expression cassette contains more than one copy (e.g., two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 20 and, thus, when aligned to SEQ ID NO: 20, there are one or more mismatches. In certain embodiments, a miR-183 target sequence comprises a sequence having at least 1, 2, 3, 4, 5, 6, 7, 8, 9, or 10 mismatches when aligned to SEQ ID NO: 20, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 20, i.e., a sequence that lacks at least 1, 2, 3, 4, 5, 6, 7, 8, 9, or 10 nucleotides at either or both the 5′ or 3′ ends of SEQ ID NO: 20. In certain embodiments, the expression cassette or vector genome comprises a transgene and one miR-182 target sequence. In yet other embodiments, the expression cassette or vector genome comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence. As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different. In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence. In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

See, PCT/US19/67872, filed Dec. 20, 2019, and now published as WO 2020/132455, which is incorporated by reference herein and which claims priority to US Provisional U.S. Patent Application No. 62/783,956, filed Dec. 21, 2018, which are hereby incorporated by reference. See, also, U.S. Patent Application No. 63/023,593, filed May 12, 2020, U.S. Patent Application No. 63/038,488, filed Jun. 12, 2020, U.S. Patent Application No. 63/043,562, filed Jun. 24, 2020, U.S. Patent Application No. 63/079,299, filed Sep. 16, 2020, and U.S. Provisional Patent Application No. 63/152,042, filed Feb. 22, 2021, and International Patent Application No. PCT/US21/32003 all of which are hereby incorporated by reference.

Optionally, the expression cassette may include UBE3A coding sequence encoding for a UBE3A protein which is a fusion protein comprising a signal peptide and/or an uptake peptide, as described herein. In certain embodiments, a signal peptide and/or an uptake peptide are located at either 5′ or 3′ of the UBE3A coding sequence.

Vector genomes comprising engineered hUBE3A-isoform 1 coding sequences are provided herein, e.g., in SEQ ID NO: 1 (hSyn.hUbe3a-1.GSco.4×miRNA183.SV40 (with miR183 target sequences)) and SEQ ID NO: 3 (hSyn.hUbe3a-1.GSco.SV40, without miR)).

Vector genomes comprising an engineered hUBE3A-isoform 2 coding sequences are illustrated herein, e.g., in SEQ ID NO: 5 (hSyn.hUbe3a-2.GSco.4×miRNA183.SV40 (with miR183 target sequences)) and SEQ ID NO: 7 (hSyn.hUbe3a-2.GSco.SV40, without miR)).

It should be understood that the compositions in the expression cassettes described herein are intended to be applied to the compositions and methods described across the Specification.

### Vectors

In certain embodiments, the expression cassette encoding UBE3A is delivered to neurons by a vector or a viral vector, of which many are known and available in the art. In one embodiment, provided is a vector comprising the UBE3A gene as described herein. In one embodiment, provided is a vector comprising an expression cassette as described herein. In one embodiment, the vector is a non-viral vector. In a further embodiment, the non-viral vector is a plasmid. In another embodiment, the vector is a viral vector. Viral vectors include any virus suitable for gene therapy, including but not limited to a bocavirus, adenovirus, adeno-associated virus (AAV), herpes virus, lentivirus, retrovirus, or parvovirus. However, for ease of understanding, the adeno-associated virus is referenced herein as an exemplary virus vector. Thus, in one embodiment, an adeno-associated viral vector comprising a nucleic acid sequence one or more elements of expression cassette operatively linked to regulatory elements therefor is provided.

A “vector” as used herein is a biological or chemical moiety comprising a nucleic acid sequence which can be introduced into an appropriate target cell for replication or expression of a nucleic acid sequence. Examples of a vector include but are not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule having an exogenous or heterologous engineered nucleic acid encoding a functional gene product, which can then be introduced into an appropriate target cell. Such vectors preferably have one or more origins of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization, or quantification of the vectors are available to one of skill in the art.

As used herein, a recombinant viral vector is any suitable viral vector which targets the desired cell(s). Thus, the recombinant viral vectors described herein preferably target one or more of the cells and tissues affected by Angelman syndrome, including cells of the central nervous system (e.g., brain). The examples provide illustrative recombinant adeno-associated viruses (rAAV). However, other suitable viral vectors may include, e.g., a recombinant adenovirus, a recombinant parvovirus such a recombinant bocavirus, a hybrid AAV/bocavirus, a recombinant herpes simplex virus, a recombinant retrovirus, or a recombinant lentivirus. In preferred embodiments, these recombinant viruses are replication-defective.

A “replication-defective” virus or viral vector refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

“Plasmid” or “plasmid vector” generally is designated herein by a lower-case p preceded and/or followed by a vector name. Plasmids, other cloning and expression vectors, properties thereof, and constructing/manipulating methods thereof that can be used in accordance with the present invention are readily apparent to those of skill in the art. In one embodiment, the elements of a vector genome as described herein or the expression cassette as described herein are engineered into a suitable genetic element (a vector) useful for generating viral vectors and/or for delivery to a host cell, e.g., naked DNA, phage, transposon, cosmid, episome, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

The term “transgene” or “gene of interest” as used interchangeably herein means an exogenous and/or engineered protein-encoding nucleic acid sequence that is under the control of a promoter and/or other regulatory elements in an expression cassette, rAAV genome, recombinant plasmid or production plasmid, vector, or host cell described in this specification.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence with which the protein or nucleic acid in question is not found in the same relationship to each other in nature.

As used herein, the term “host cell” may refer to the packaging cell line in which a vector (e.g., a recombinant AAV) is produced from a production plasmid. In the alternative, the term “host cell” may refer to any target cell in which expression of a gene product described herein is desired. Thus, a “host cell,” refers to a prokaryotic or eukaryotic cell (e.g., human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. In a further embodiment, the term “host cell” is a neuron, e.g., a neuron of the CNS.

As used herein, the term “target cell” refers to any target cell in which expression of a heterologous nucleic acid sequence or protein is desired. In certain embodiments, the target cell is a neuron of the CNS, in particular a neuron with a mutated or defective maternal UBE3A allele or a neuron that lacks UBE3A expression.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Further, the vector genome contains regulatory sequences which direct expression of the gene products. Suitable components of a vector genome are discussed in more detail herein. In one example, a “vector genome” contains, at a minimum, from 5′ to 3′, a vector-specific sequence, a nucleic acid sequence encoding UBE3A operably linked to regulatory control sequences which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids. Lentivirus long terminal repeats may be utilized where packaging into a lentiviral vector is desired. Similarly, other terminal repeats (e.g., a retroviral long terminal repeat), or the like may be selected.

Vector genomes encoding UBE3A isoform 1 provided herein include, e.g., SEQ ID NO: 1 (AAV2-5′ ITR-hSyn.hUbe3a-1.GSco.4×miRNA183.SV40-AAV2-3′ ITR), SEQ ID NO: 3 (AAV2-5′ ITR-hSyn.hUbe3a-1.GSco.SV40-AAV2-3′ ITR).

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV nuclease (e.g., DNase)-resistant particle having an AAV protein capsid into which is packaged expression cassette flanked by AAV inverted terminal repeat sequences (ITRs) for delivery to target cells. A nuclease-resistant recombinant AAV (rAAV) indicates that the AAV capsid has fully assembled and protects these packaged vector genome sequences from degradation (digestion) during nuclease incubation steps designed to remove contaminating nucleic acids which may be present from the production process. In many instances, the rAAV described herein is DNase resistant.

In the examples below, the clade F adeno-associated virus is AAVhu68. See, WO 2018/160582, which is incorporated by reference herein in its entirety. In other embodiments, another AAV capsid is selected from a different clade, e.g., clade A, B, C, D, or E, or from an AAV source outside of any of these clades. For example, another suitable capsid is AAVrh91. See WO 2020/223231, published Nov. 5, 2020, U.S. Patent Application No. 63/065,616, filed Aug. 14, 2020, and U.S. Patent Application No. 63/109,734, filed Nov. 4, 2020, International Patent Application No. PCT/US21/55436, which are incorporated herein by reference. In certain embodiments, AAV capsids having reduced capsid deamidation may be selected. See, e.g., PCT/US19/19804 and PCT/US18/19861, both filed Feb. 27, 2019 and incorporated by reference in their entireties. See also, PCT/US20/030266, filed Apr. 29, 2020, now published WO2020/223231, and International Application No. PCT/US21/45945, filed Aug. 13, 2021, which are incorporated herein by reference.

In other embodiments, the source of the AAV capsid may be one of any of the dozens of naturally occurring and available adeno-associated viruses, as well as engineered or artificial AAVs. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (AAVrh10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8 and AAVAnc80. See, e.g., WO 2005/033321, which is incorporated herein by reference. In one embodiment, the AAV capsid is an AAV9 capsid or variant thereof. In certain embodiments, the capsid protein is designated by a number or a combination of numbers and letters following the term “AAV” in the name of the rAAV vector. See, also PCT/US19/19804 and PCT/US19/19861, each entitled “Novel Adeno-Associated Virus (AAV) Vectors, AAV Vectors Having Reduced Capsid Deamidation And Uses Therefor” and filed Feb. 27, 2019, which are incorporated by reference herein in their entireties.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV” and “recombinant AAV vector” are used interchangeably, mean, without limitation, an AAV comprising a capsid protein and a vector genome packaged therein, wherein the vector genome comprising a nucleic acid heterologous to the AAV. rAAV includes “pseudotyped rAAV”, wherein the viral vector contains a vector genome containing the inverted terminal repeat of one AAV (e.g., AAV2) packaged into the capsid of a different AAV capsid protein. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 15 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

In one aspect, provided herein is and AAV vector which comprises an AAV capsid and an expression cassette, wherein the expression cassette comprises a nucleic acid sequence encoding one more elements of a UBE3A gene and regulatory elements that direct expression of the elements of the UBE3A gene in a host cell. The AAV vector also comprises AAV ITR sequences.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (ΔITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the nucleic acid sequences encoding the gene product(s) and any regulatory sequences, and an AAV 3′ ITR. However, other configurations of these elements may be suitable. In one embodiment, a self-complementary AAV is provided. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external “a” element is deleted. The shortened ITR is reverted back to the wild-type length of 145 base pairs during vector DNA amplification using the internal A element as a template. In other embodiments, the full-length AAV 5′ and 3′ ITRs are used.

In one embodiment, the regulatory sequences are selected such that the total rAAV vector genome is about 2.0 to about 5.5 kilobases in size. In one embodiment, the regulatory sequences are selected such that the total rAAV vector genome is about 2.9 to about 5.5 kilobases in size. In one embodiment, the regulatory sequences are selected such that the total rAAV vector genome is about 2.9 kb in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al., Mol Ther, January 2010, 18(1):80-6, which is incorporated herein by reference.

In certain embodiments, provided herein is a rAAV useful as CNS-directed therapy for treatment of a subject having Angelman syndrome (AS), wherein the rAAV comprises an AAV capsid, and a vector genome packaged therein, said vector genome comprising: (a) an AAV 5′ inverted terminal repeat (ITR); (b) a sequence encoding UBE3A which is operably linked to regulatory elements which direct expression thereof in a host cell; (c) regulatory elements which direct expression; and (d) an AAV 3′ ITR. In certain embodiments, provided herein is a rAAV useful as CNS-directed therapy for treatment of a subject having Angelman syndrome (AS), wherein the rAAV comprises an AAV capsid, and a vector genome packaged therein, said vector genome comprising: (a) an AAV 5′ inverted terminal repeat (ITR); (b) a sequence encoding UBE3A which is operably linked to regulatory elements which direct expression thereof in a host cell; (c) optionally a peptide/s (e.g., signal peptide and/or uptake peptide); (d) regulatory elements which direct expression; and (e) an AAV 3′ ITR. In one embodiment, the rAAV has a tropism for a cell of the CNS (e.g., an rAAV bearing an AAVhu68 capsid or an AAVrh91 capsid), and/or contains a neuron-specific expression control elements (e.g., a synapsin promoter). In one aspect, a construct is provided which is a vector (e.g., a plasmid) useful for generating viral vectors. In one embodiment, the AAV 5′ ITR is an AAV2 ITR and the AAV 3′ITR is an AAV2 ITR. In one embodiment, the rAAV comprises an AAV capsid as described herein. In one embodiment, the rAAV comprises an AAVhu68 capsid. In other embodiments, the rAAV comprises an AAVrh91 capsid. SEQ ID NO: 18 provides the encoded amino acid sequence of the AAVrh91 vp1 protein.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug. 10. pii: S1525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164.; Li L et al. Production and characterization of novel recombinant adeno-associated virus replicative-form genomes: a eukaryotic source of DNA for gene transfer. PLoS One. 2013 Aug. 1; 8(8):e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl:S80-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, and WO 2017/100674 entitled “Scalable Purification Method for AAV1”, which are incorporated by reference herein. In brief, the method for separating rAAV particles having packaged genomic sequences from genome-deficient AAV intermediates involves subjecting a suspension comprising recombinant AAV9 or AAV viral particles and AAV capsid intermediates to fast performance liquid chromatography, wherein the AAV9 viral particles and AAV intermediates are bound to a strong anion exchange resin equilibrated at a pH of about 10.2 for rAAV9 or about 9.8 for AAV1, and subjected to a salt gradient while monitoring eluate for ultraviolet absorbance at about 260 and about 280. In this method, the AAV full capsids are collected from a fraction which is eluted when the ratio of A260/A280 reaches an inflection point. In one example, for the affinity chromatography step, the diafiltered product may be applied to an AAV-specific resin that efficiently captures the selected AAV serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or Coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2, and vp3 of capsid protein are also available. See, e.g., Vamseedhar Rayaprolu et al., Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

It should be understood that the compositions in the vectors described herein are intended to be applied to other compositions and methods described across the Specification.

### Compositions

Provided is an aqueous suspension suitable for administration to treat AS in a subject in need thereof, said suspension comprising an aqueous suspending liquid and vector comprising an engineered nucleic acid sequence encoding a UBE3A gene operatively linked to regulatory elements therefor as described herein. In one embodiment, a therapeutically effective amount of said vector is included in the suspension.

### Nucleic Acids

In certain embodiments, the pharmaceutical composition comprises an expression cassette comprising a nucleic acid sequence encoding UBE3A isoform 1 and a non-viral delivery system. This may include, e.g., naked DNA, naked RNA, an inorganic particle, a lipid or lipid-like particle, a chitosan-based formulation and others known in the art and described for example by Ramamoorth and Narvekar, as cited above). In other embodiments, the pharmaceutical composition is a suspension comprising the expression cassette comprising the UBE3A gene in a viral vector system. In certain embodiments, the pharmaceutical composition comprises a non-replicating viral vector. Suitable viral vectors may include any suitable delivery vector, such as, e.g., a recombinant adenovirus, a recombinant lentivirus, a recombinant bocavirus, a recombinant adeno-associated virus (AAV), or another recombinant parvovirus. In certain embodiments, the viral vector is a recombinant AAV for delivery of UBE3A isoform 1 to a patient in need thereof. In certain embodiments, the viral vector is a recombinant AAV for delivery of UBE3a isoform 3 to a patient in need thereof.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

In one embodiment, the suspension further comprises a surfactant, preservative, excipients, and/or buffer dissolved in the aqueous suspending liquid. In one embodiment, the buffer is PBS. Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. A suitable surfactant, or combination of surfactants, may be selected from among Poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. The pH may be in the range of 6.5 to 8.5, or 7 to 8.5, or 7.5 to 8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other routes of delivery.

Additionally provided is a pharmaceutical composition comprising a pharmaceutically acceptable carrier and a vector comprising a nucleic acid sequence encoding one or more components of a UBE3A operatively linked to regulatory elements therefor as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered UBE3A transgene may be formulated for delivery either encapsulated in a lipid particle, a liposome, a vesicle, a nanosphere, or a nanoparticle or the like. In one embodiment, a therapeutically effective amount of said vector is included in the pharmaceutical composition. Suitable carriers may be readily selected by one of skill in the art in view of the indication for which the vector is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

The aqueous suspension or pharmaceutical compositions described herein are designed for delivery to subjects in need thereof by any suitable route or a combination of different routes. In one embodiment, the pharmaceutical composition comprises an expression cassette or vector described herein in a formulation buffer suitable for delivery via intracerebroventricular (ICV), intrathecal (IT), intracisternal, or intravenous (IV) routes of administration. Alternatively, other routes of administration may be selected (e.g., oral, inhalation, intranasal, intratracheal, intraarterial, intraocular, intramuscular, and other parenteral routes).

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, intracerebroventricular (icv) suboccipital/intracisternal, and/or C1-2 puncture. For example, material may be introduced for diffusion throughout the subarachnoid space by means of lumbar puncture. In another example, injection may be into the cisterna magna (intracisternal magna; ICM). Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51. As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

In one aspect, provided herein is a pharmaceutical composition comprising a vector as described herein in a formulation buffer. In certain embodiments, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹ 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10² GC per dose including all integers or fractional amounts within the range.

In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In the case of AAV viral vectors, quantification of the genome copies (“GC”) may be used as the measure of the dose contained in the aqueous suspension or pharmaceutical compositions. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR or quantitative PCR using primer/probe sets targeting specific region of the viral genome (usually poly A signal). The replication-defective virus compositions can be formulated in dosage units to contain an amount of replication-defective virus that is in the range of about 1.0×10⁹ GC to about 1.0×10¹⁵ GC, and preferably 1.0×10¹² GC to 1.0×10¹⁴ GC for a human patient. Preferably, the concentration of replication-defective virus in the formulation is about 1.0×10⁹ GC, about 5.0×10⁹ GC, about 1.0×10¹⁰ GC, about 5.0×10¹⁰ GC, about 1.0×10¹¹ GC, about 5.0×10¹¹ GC, about 1.0×10¹² GC, about 5.0×10¹² GC, about 1.0×10¹³ GC, about 5.0×10¹³ GC, about 1.0×10¹¹ GC, about 5.0×10¹⁴ GC, or about 1.0×10¹⁵ GC. Alternative or additional method for performing AAV GC number titration is via oqPCR or digital droplet PCR (ddPCR) as described in, e.g., M. Lock et al, Hum Gene Ther Methods. 2014 April; 25(2):115-25. doi: 10.1089/hgtb.2013.131. Epub 2014 Feb. 14, which is incorporated herein by reference.

It should be understood that the compositions in the pharmaceutical compositions described herein are intended to be applied to other compositions, regimens, aspects, embodiments, and methods described across the Specification

### Methods

In certain embodiments, an expression cassette, nucleic acid, or a viral or non-viral vector is used in preparing a medicament. In certain embodiments, uses of the same for treatment of Angelman syndrome in a subject in need thereof are provided.

As used herein, the term “treatment” or “treating” is defined encompassing administering to a subject one or more compounds or compositions described herein for the purposes of amelioration of one or more symptoms of UBE3A deficiency or Angelman syndrome (AS). “Treatment” can thus include one or more of reducing onset or progression of AS, preventing disease, reducing the severity of the disease symptoms, retarding their progression, removing the disease symptoms, delaying progression of disease, or increasing efficacy of therapy in a given subject.

Therapies described provide UBE3A isoform 1 expression to achieve a desired result, i.e., treatment of Angelman syndrome (AS) or one or more symptoms thereof. Such symptoms may include but are not limited to one of more of the following: intellectual disability, speech impairment, ataxia, epilepsy, seizure disorder, microcephaly, psychomotor delay, and muscular hypotonia with hyperreflexia (See e.g., Buiting K et al., Angelman syndrome-insight into a rare neurogenetic disorder, Nat Rev Neurol, 2016, 12(10): 584-593, epub Sep. 12, 2016, which is incorporated herein by reference). As described herein, a desired result may include reducing or eliminating neurophysical complications including delayed development, intellectual disability, severe speech impairment, and problems with movement and balance.

A “therapeutically effective amount” of a composition provided herein is delivered to a subject to achieve a desired result or to reach a therapeutic goal. In one embodiment, a therapeutic goal for treating AS is to restore UBE3A isoform 1 expression in a neuron, or in a population of neurons, to the functional level in a patient that is in the normal range or to the non-AS level. In another embodiment, therapeutic goal for treatment of AS is to increase the UBE3A isoform 1 expression to at least about 99%, about 95%, about 90%, about 85%, about 80%, about 75%, about 70%, about 65%, about 60%, about 55%, about 50%, about 45%, about 40%, about 45%, about 40%, about 35%, about 30% about 25%, about 20%, about 15%, about 10%, about 5%, about 2%, about 1% of the normal or non-AS level, or as compared to levels of UBE3A expression before treatment. Patients rescued by delivering UBE3A isoform 1 function to less than 100% activity levels may optionally be subject to further treatment. In another embodiment, therapeutic goals for treatment of AS are to increase the UBE3A isoform 1 expression in a percentage of target neurons, including about 60%, about 55%, about 50%, about 45%, about 40%, about 45%, about 40%, about 35%, about 30% about 25%, about 20%, about 15%, about 10%, about 5%, about 2%, or about 1% of neurons in a selected population.

In certain embodiments, provided herein is a method of treating AS by administering to a subject in need thereof an expression cassette, vector, or rAAV that provides UBE3A isoform 1 results in expression of functional UBE3A isoform 1 in a neuron. In certain embodiments, the method includes delivering a nucleic acid sequence which expresses UBE3A isoform 1 (amino acid sequence of SEQ ID NO: 2).

In certain embodiments, provided herein is a method of enzyme replacement by administering to a subject in need thereof of an expression cassette, vector, or rAAV that provides UBE3a isoform 1 resulting in expression of functional UBE3a isoform 1 in a neuron.

In certain embodiments, the method includes delivering a nucleic acid sequence which express UBE3a isoform 1 (amino acid sequence of SEQ ID NO: 2). In certain embodiments, provided herein is a method of enzyme replacement by administering to a subject in need thereof of an expression cassette, vector, or rAAV that provides UBE3a isoform 3 resulting in expression of functional UBE3a isoform 3 in a neuron. In certain embodiments, the method includes delivering a nucleic acid sequence which express UBE3a isoform 3 (amino acid sequence of SEQ ID NO: 21)

The gene therapy described herein, whether viral or non-viral, may be used in conjunction with other treatments (secondary therapy), i.e., the standard of care for the subject's (patient's) diagnosis and condition. As used herein, the term “secondary therapy” refers to the therapy that could be combined with the gene therapy described herein for the treatment of AS. In some embodiments, the gene therapy described herein is administered in combination with one or more secondary therapies for the treatment of AS, such as administering an anticonvulsant or dietary restriction (e.g., ketogenic and low glycemic). The secondary therapy may be any therapy which helps prevent, arrest or ameliorate these symptoms of AS. The secondary therapy can be administered before, concurrent with, or after administration of the compositions described above. Subjects may be permitted to continue their standard of care treatment(s) prior to and concurrently with the gene therapy treatment at the discretion of their caring physician. In the alternative, the physician may prefer to stop standard of care therapies prior to administering the gene therapy treatment and, optionally, resume standard of care treatments as a co-therapy after administration of the gene therapy. In another embodiment, the gene therapy described herein may be combined with genotypic analysis or genetic screening, which is routine in the art and may include the use of PCR to identify one or more mutations in the nucleic acid sequence of the UBE3A gene. As discussed above, subjects showing symptoms of AS early in life (e.g., 1-3 months) as well as subjects diagnosed with AS later in life are the intended recipients of the compositions and methods described herein.

By “administering” or “route of administration” is delivery of composition described herein, with or without a pharmaceutical carrier or excipient, of the subject. Routes of administration may be combined, if desired. In some embodiments, the administration is repeated periodically. Sequential administration may imply a time gap of multi-administration from intervals of days, weeks, months or years. In one embodiment, the compositions described herein are administered to a subject in need for one or more times. In one embodiment, the administrations are days, weeks, months or years apart. In one embodiment, one, two, three or more re-administrations are permitted. Such re-administration may be with the same type of vector, or a different vector. In a further embodiment, the vectors described herein may be used alone, or in combination with the standard of care for the patient's diagnosis and condition. The nucleic acid molecules and/or vectors described herein may be delivered in a single composition or multiple compositions. Optionally, two or more different AAV may be delivered, or multiple viruses [see, e.g., WO 2011/126808 and WO 2013/049493].

In one embodiment, the expression cassette, vector, or other composition described herein for gene therapy is delivered as a single dose per patient. In one embodiment, the subject is delivered a therapeutically effective amount of a composition described herein. As used herein, a “therapeutically effective amount” refers to the amount of the expression cassette or vector, or a combination thereof.

In one embodiment, the expression cassette is in a vector genome delivered in an amount of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

In certain embodiments, a regimen may involve additional treatment that includes a composition comprising a gene editing system. See, e.g., U.S. Patent Application No. 63/016,712, filed Apr. 28, 2020 and U.S. Patent Application No. 63/118,299, filed Nov. 25, 2020, entitled “Compositions and uses for Treatment of Angelman Syndrome”. This treatment may be prior to treatment with the gene replacement therapy described herein and may utilize vectors having different capsids than were utilized for the initial treatment. Still other combinations of AAV capsids may be selected by one skilled in the art.

In certain embodiments, a therapeutic regimen may involve co-expression of UBE3A isoform 1 with UBE3A isoform 3. In certain embodiments, a therapeutic may involve co-therapy of the AAV.hUBE3A-isoform 1 and hUBE3A enzyme replacement therapy (e.g., with isoform 3 and/or isoform 1 enzyme). In certain embodiments, a therapeutic regimen may involve co-therapy with an AAV.hUBE3A-isoform 1 gene therapy vector and an immunomodulatory regimen. Such an immunomodulatory regimen may include, e.g., but are not limited to immunosuppressants such as, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25−) or CD3-directed antibodies, anti-IL-2 antibodies, cyclosporin, tacrolimus, sirolimus, IFN-β, IFN-γ, an opioid, or TNF-α (tumor necrosis factor-alpha) binding agent. In certain embodiments, the immunosuppressive therapy may be started prior to the gene therapy administration. Such therapy may involve co-administration of two or more drugs, the (e.g., prednisolone, micophenolate mofetil (MMF) and/or sirolimus (i.e., rapamycin)) on the same day. One or more of these drugs may be continued after gene therapy administration, at the same dose or an adjusted dose. Such therapy may be for about 1 week, about 15 days, about 30 days, about 45 days, 60 days, or longer, as needed. Still other co-therapeutics may include, e.g., anti-IgG enzymes, which have been described as being useful for depleting anti-AAV antibodies (and thus may permit administration to patients testing above a threshold level of antibody for the selected AAV capsid), and/or delivery of anti-FcRN antibodies which is described, e.g., in U.S. Provisional Patent Application No. 63/040,381, filed Jun. 17, 2020, entitled “Compositions and Methods for Treatment of Gene Therapy Patients”, and/or one or more of a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.

Generally, the methods include administering to a mammalian subject in need thereof, a pharmaceutically effective amount of a composition comprising a recombinant adeno-associated virus (AAV) carrying a nucleic acid sequence encoding one or more elements of a UBE3A gene replacement (expression) system under the control of regulatory sequences, and a pharmaceutically acceptable carrier. In one embodiment, such a method is designed for treating, retarding or halting progression of AS in a mammalian subject.

In one embodiment, provided is a method of treating AS by administering to a subject in need the vector, the rAAV, the aqueous suspension, or the pharmaceutical composition as described in the present specification. In one embodiment, a rAAV is delivered about 1×10¹⁰ to about 1×10¹⁵ genome copies (GC)/kg body weight. In certain embodiments, the subject is human. In one embodiment, the rAAV is administered more than one time. In a further embodiment, the rAAV is administered days, weeks, months or years apart.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

Angelman syndrome (AS) is a rare neurodevelopmental disorder affecting about half of a million individuals worldwide. AS is characterized by intellectual and physical disability, seizures and impairments in sleep, and gut function. Many of these deficits are caused by the loss of the maternally inherited ubiquitin protein ligase E3A (UBE3A) allele. At present, therapeutic options for AS are limited. AAV-based gene replacement therapy represents a promising strategy for restoring UBE3A isoform expression and mitigating AS severity. However, the UBE3A gene encodes three isoforms and it is currently unclear which UBE3A isoform would make the most effective candidate. We address this uncertainty by comparing the efficacy of AAV vectors delivering engineered human UBE3A isoforms in a mouse model of AS (UBE3A^{m-/p+}). Western blotting and immunohistochemical analyses indicated that intracerebroventricular injections of AAV-UBE3A human isoform 1 and 2 vectors into neonatal control and AS mice resulted in robust protein expression. Isoform 1 replacement significantly improved gait, nest-building ability, and motor coordination in a dose-dependent manner in AS mice. Unlike isoform 1, isoform 2 further impaired nest-building ability and motor coordination in AS mice. The toxicology studies in nonhuman primates suggested that a high dose of AAV-UBE3A isoform 1 vector injected into the cisterna magna had no significant adverse effects. Taken together, these data suggest that the AAV-UBE3A isoform 1 vector is most therapeutically effective with a promising safety profile. These preclinical findings represent an important step forward in the development of gene replacement therapy for AS.

### Example 1—Engineered hUBE3A Isoform 1 and Engineered hUBE3A Isoform 2 Coding Sequences Under the Direction of a Modified Human Synapsin Promoter

For a first set of studies in mice, we engineered a series of plasmids containing an engineered human UBE3A isoform 1 (SEQ ID NO: 9) or UBE3A isoform 2 (SEQ ID NO: 10) transgene that were under the control of a modified human synapsin (hSyn) promotor (SEQ ID NO: 12). The SV40 polyadenylation sequence (SEQ ID NO: 13) was included to promote transcript stability. Promoter-transgene-SV40 constructs were flanked by inverted terminal repeats (ITR). Plasmids were packaged into PHP.B, an AAV9 variant that robustly transduces neurons and glia of the central nervous system of C57BL6/J mice.

The coding sequence of the modified human synapsin promoter is provided in SEQ ID NO: 12.

The resulting vector genomes are reproduced in SEQ ID NO: 3 (hSyn.hUbe3a-1.GSco.SV40) and SEQ ID NO: 7 (isoform 2 hSyn.hUbe3a-2.GSco.SV40).

The AAVPHP.B capsid (U.S. Pat. No. 9,585,971) is generated in a packaging host cell using triple transfection techniques in a trans plasmid comprising AAV2 rep coding sequences and the PHP.B VP1 coding sequence, co-transfected with the cis plasmid containing the vector genome and a trans plasmid expressing the necessary adenovirus helper functions not provided by the packaging host cell.

### Example 2—rAAV-Mediated Delivery of hUBE3A to Mice and NHP

A. Expression Testing of hUBE3A Isoform 1 and 2 in Adult Mouse Brain

We identified the highest expressing AAV-PHP.B-synapsin-UBE3A isoform 1 and AAV-PHP.B-synapsin-UBE3A-isoform 2 vectors by injecting them (intracerebroventricular-ICV) into the brains of wild type adult mice and quantifying transgene expression by Western blot (FIG. 8).

Method: 5×10¹¹ GC/mouse delivered by retro orbital (IV) injection in adult wild type WT (UBE3A^{m+/p+}) and UBE3A KO/null (UBE3A^{m-/p+}) littermates. Brains harvested 14 days post injection for Western blotting. cDNAs were then subcloned into plasmids with fully intact ITRs for optimal expression.

B. Determining the Most Therapeutically Efficacious AAV-UBE3A Isoform Vector

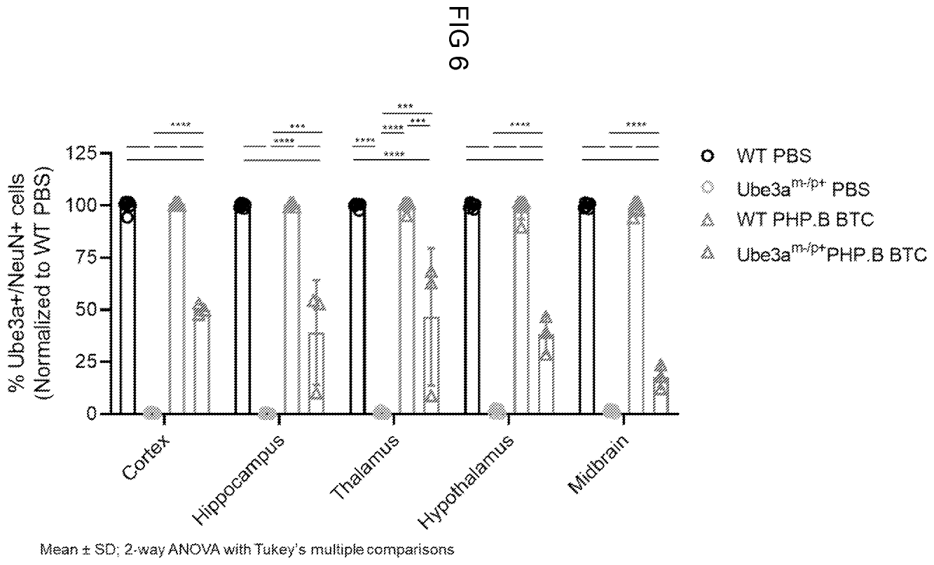

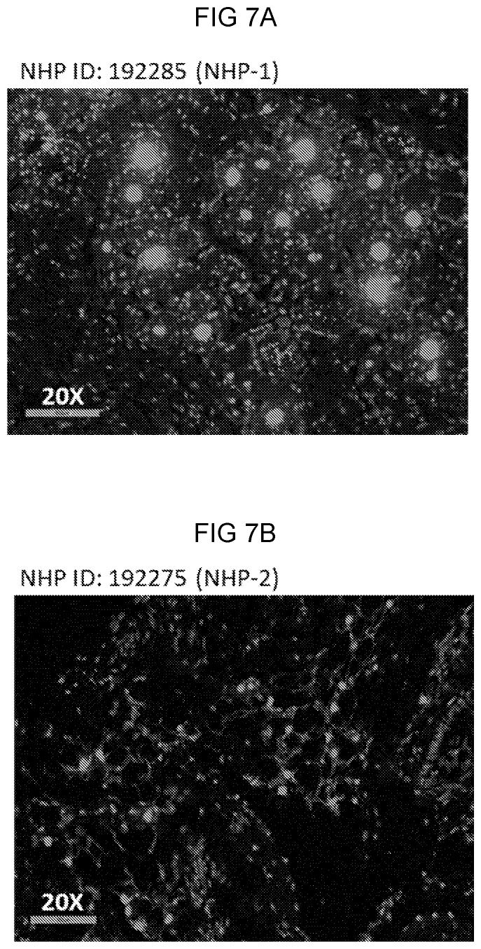

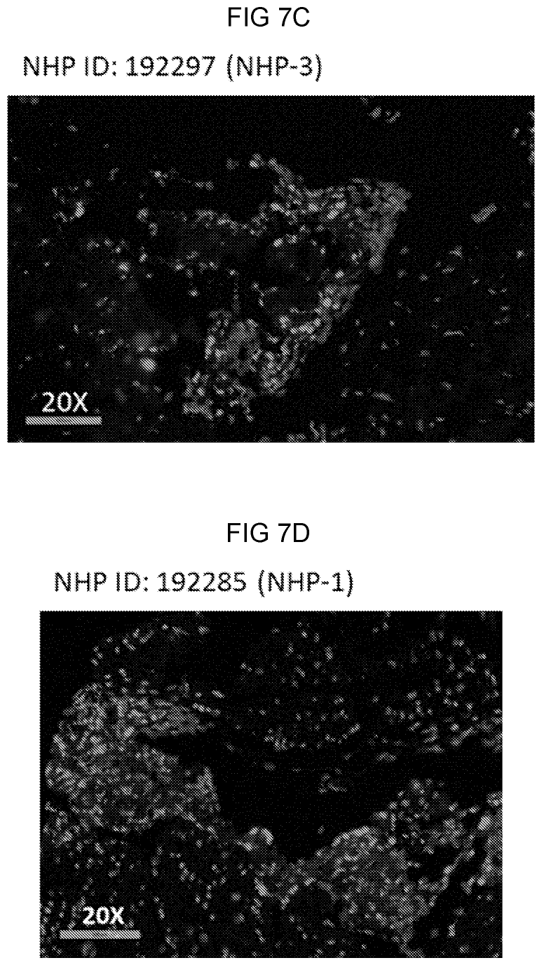

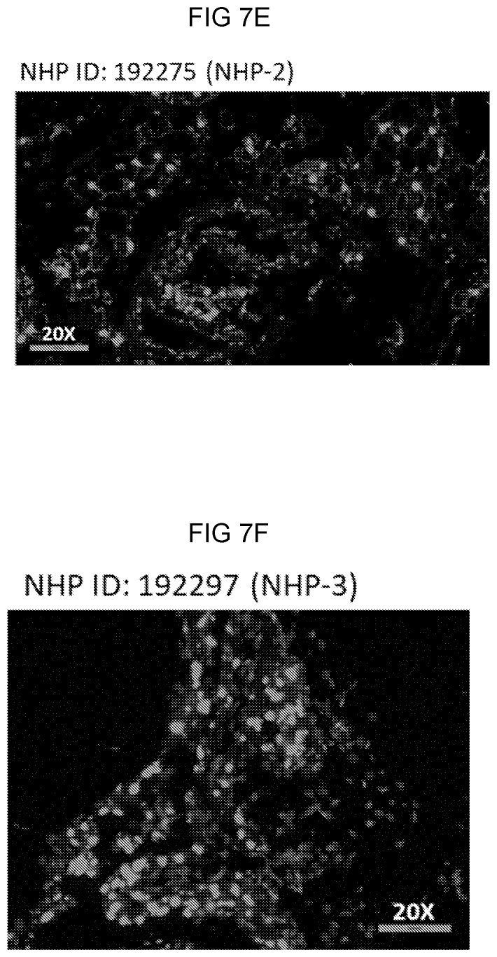

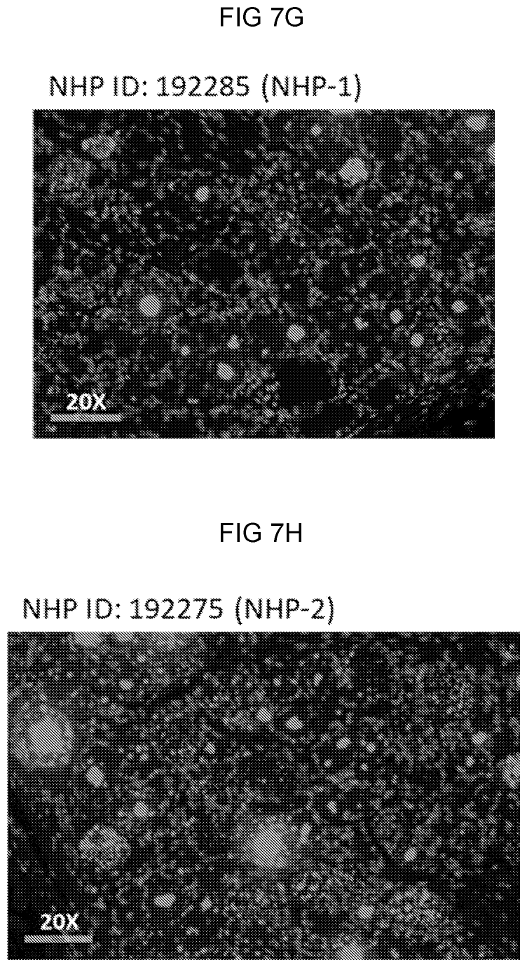

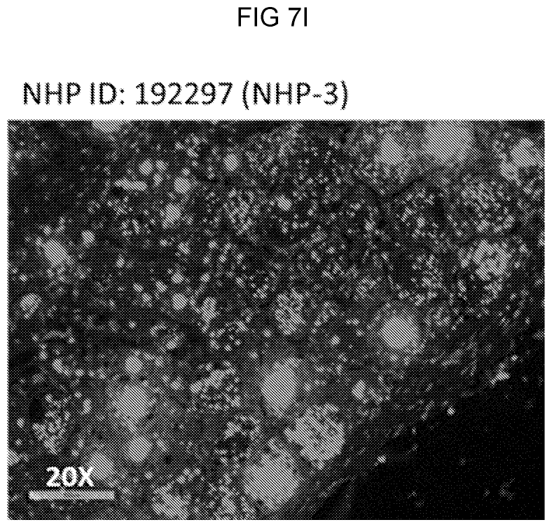

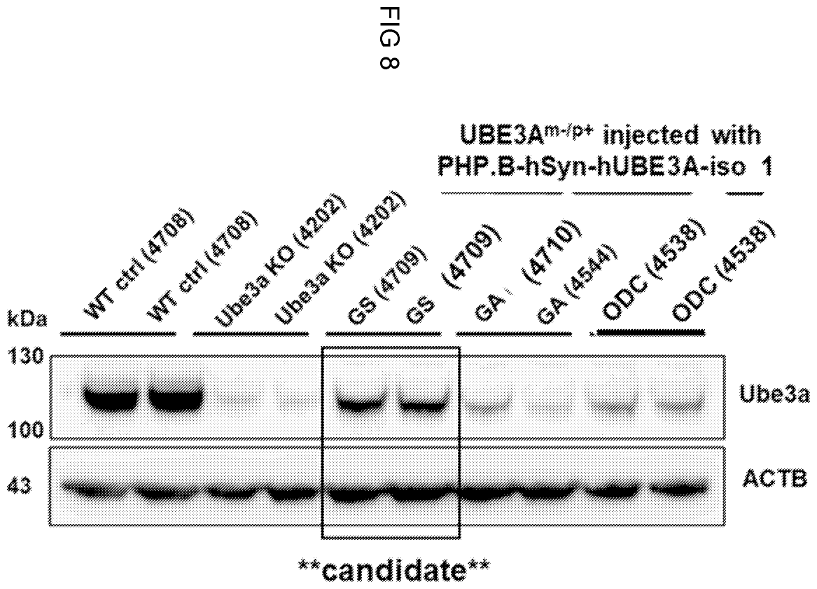

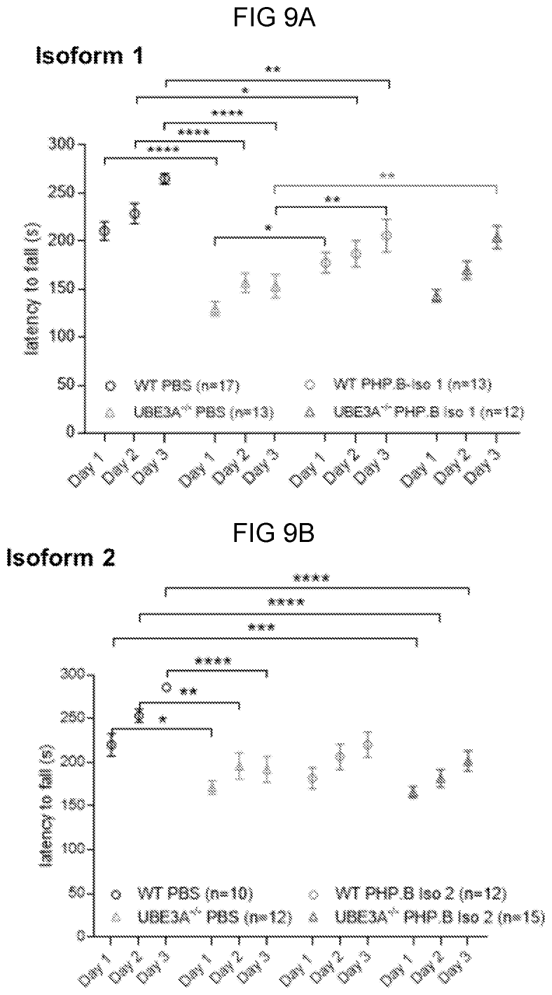

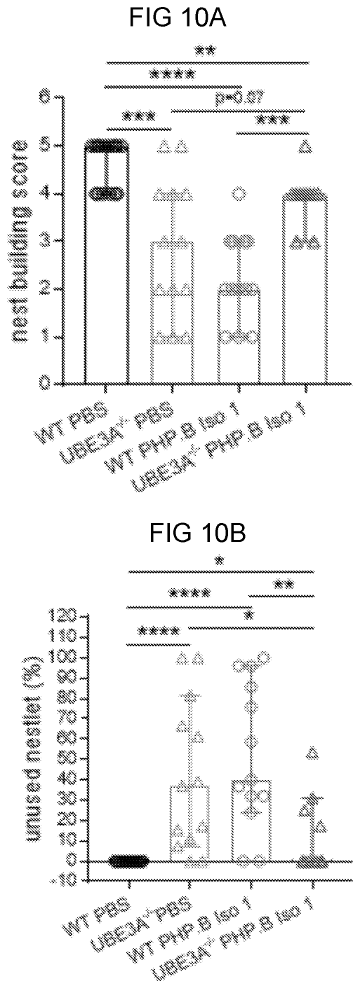

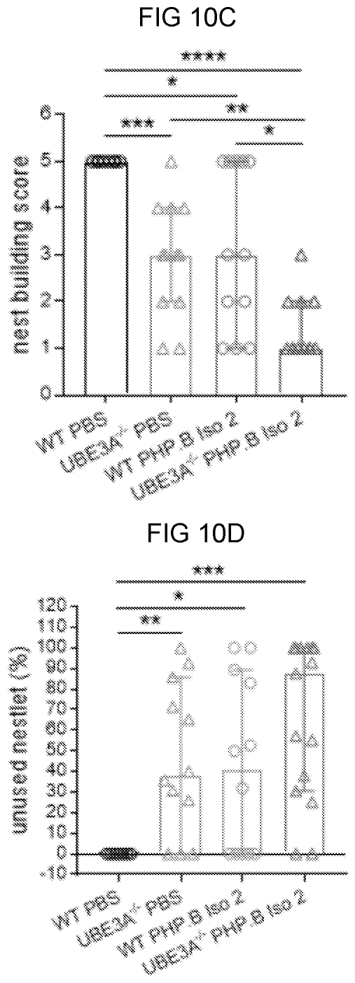

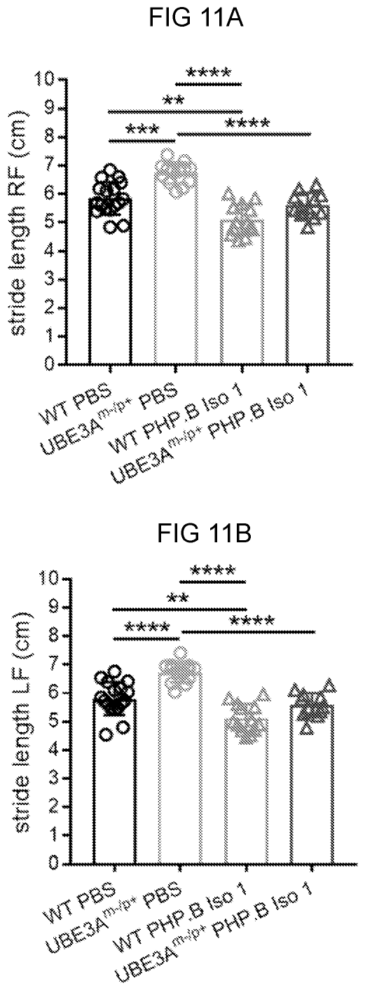

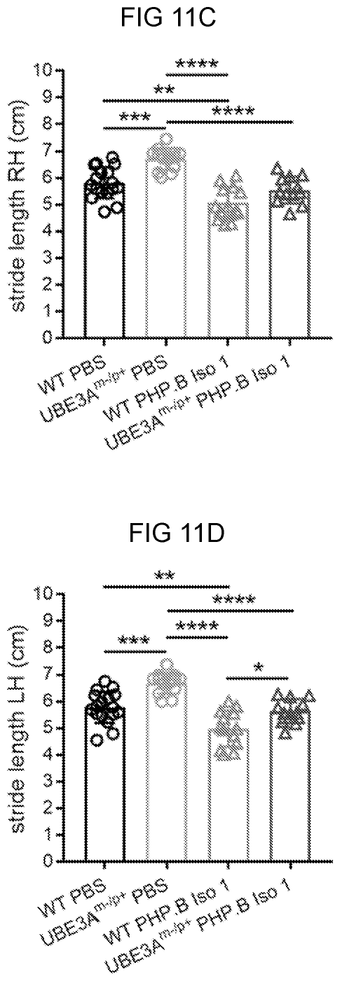

We directly compared the ability of the highest expressing AAV-PHP.B-synapsin-UBE3A-isoform 1 (isoform 1) and AAV-PHP.B-synapsin-UBE3A-isoform 2 (isoform 2) vectors to rescue motor and behavioral deficits in a mouse model of AS. Neonatal wild type or AS (UBE3A^{m-/p+}) mice were injected intracerebroventricularly (ICV) with either isoform 1 or isoform 2 vectors at a dose of 1×10¹¹ genome copies per animal. Behavioral testing (8-10 weeks of age), test order: (1) catwalk, (2) locomotor activity, (3) rotarod, (4) nest building. After two months, AS mice injected with isoform 1, but not isoform 2, showed statistically significant improvements in gait (stride length FIGS. 11A to 11D), nest building ability (FIGS. 10A and 10B) and motor coordination (FIGS. 9A and 9B). In fact, expression of isoform 2 worsened deficits in nest building ability in AS mice (FIGS. 10C and 10D). Injection of isoform 1 at a lower dose of 1×10¹⁰ genome copies per animal was less efficacious, but did improve nest building ability in AS mice. We also observed that overexpression of isoform 1 or 2 in wild type mice had adverse effects on several behavioral domains indicating the importance of tight control of UBE3A isoform expression. In summary, head-to-head testing indicated that nuclear UBE3A isoform 1 vector had a greater therapeutic capacity than cytoplasmic isoform 2 vector in reducing behavioral deficits in AS mice. The expression and localization of engineered human UBE3A isoform 1 protein was confirmed by immunofluorescence images by co-staining with neuronal marker NeuN and nuclei in sagittal brain sections (i.e., cortex, hippocampus, thalamus, hypothalamus, midbrain) from wild type, UBE3A^{m-/p+} and treated UBE3A^{m-/p+} mice. The fraction (percentage) of UBE3A isoform 1 (FIGS. 5 and 6) and UBE3A isoform 2 (FIG. 7) protein positive neurons in different brain regions was determined and quantified using Visiopharm software (FIGS. 5 and 6).

At the 1×10¹⁰ GC dose, AAV-PHP.B-hSyn-UBE3A-isoform 1 dosing achieved ˜50% of wild type endogenous UBE3A isoform expression, did not significantly impact behavioral deficits in locomotor activity, motor coordination or nest building, and improved some gait abnormalities. FIG. 5 shows quantification of UBE3A protein positive neurons in treated UBE3A^{m-/p+} mice (statistical analysis: mean±SD, unpaired student t test). Treatment comprised administration of AAV-PHP.B-hSyn-UBE3A-isoform 1 at a dose of 1×¹⁰′° GC/animal via intracerebroventricular (ICV).

At the higher dose of 1×10¹¹ GC, AAV-PHP.B-hSyn-UBE3A-isoform 1 dosing achieved ˜100% of wild type endogenous UBE3A isoform expression, significantly improved nest building ability (FIG. 10 A). Treated UBE3A^{m-/p+} mice used significantly more nestlet material to build their nests (FIG. 10B), performed according to the method of Deacon R M J, 2006, Assessing nest building in mice, Nat Protoc 1(3):1117-9. UBE3A-isoform 1 dosing had no impact on hypoactivity but normalized to a previously published gait abnormality in UBE3A^{mp+} mice (stride length assessed as described in Heck et al., Analysis of cerebellar function in Ube3a-deficient mice reveals genotype-specific behaviors, 2008, Hum Mol Genetics, 17(14): 2181-2189; epub Apr. 15, 2008). UBE3A-isoform 1 dosing (FIG. 9A), but not isoform 2 dosing (FIG. 9B) significantly reduced motor coordination deficits in UBE3A^{m-/+} mice. FIG. 6 shows quantification of UBE3A protein positive neurons in treated UBE3A^{m-/+} mice (statistical analysis: unpaired t test). Treatment comprised of administration of AAV-PHP.B-hSyn-UBE3A-isoform 1 at a dose of 1×10¹¹ GC/animal via intracerebroventricular (ICV). The quantification of protein expression from FIG. 5 is Summarized in table 1 below.

TABLE 1

| Brain Section | UBE3A-1-percent positive neurons |
|---|---|
| Cortex | ~68% |
| Hippocampus | ~63% |
| Thalamus | ~58% |
| Hypothalamus | ~42% |
| Midbrain | ~47% |

Therapeutic effects of UBE3A-isoform replacement: Isoform 1: (i) normalizes stride length (clinically relevant gait defect), (ii) improves motor coordination, (iii) improves nest building behavior, (iv) has no impact on locomotor hypoactivity in UBE3 deficient (UBE3A^{m-/p+}) mice. Isoform 2: (i) impairs nest building behavior and (ii) has no impact on locomotor hypoactivity (like isoform 1) or motor coordination.

Additionally, the data, as described above, shows that robust UBE3A isoform 1 protein expression was seen in a large fraction of neurons in several brain regions. UBE3A isoform 1 protein expression showed dose dependence with a greater percentage of UBE3A positive neurons in animals treated with 1×10¹¹ GC/animal versus 1×10¹⁰ GC/animal.

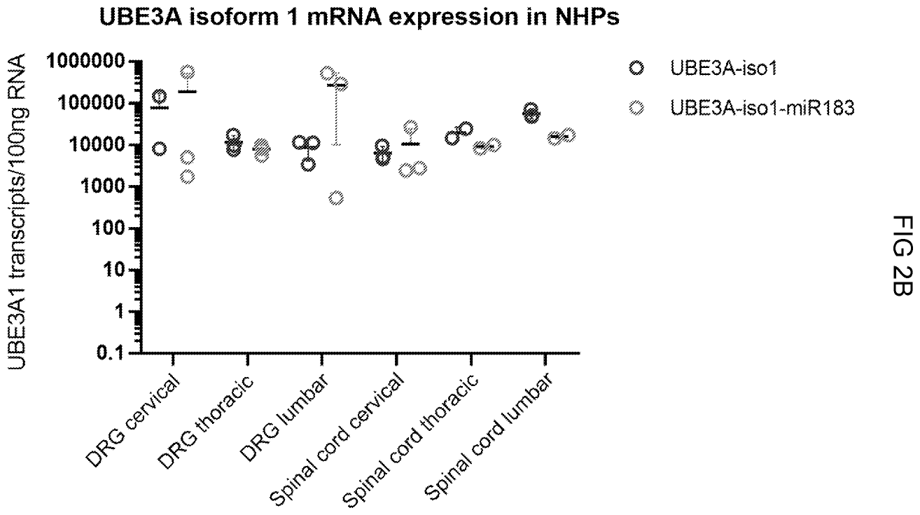

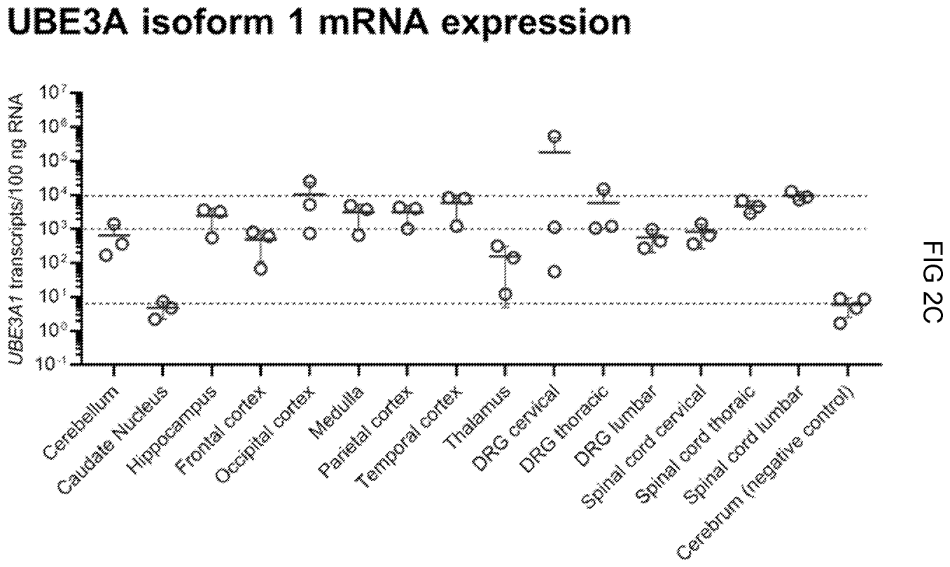

Preclinical studies in AS mice indicated that the AAV-PHP.B-synapsin-UBE3A-isoform 1 expressed well and mitigated motor and behavioral deficits in AS mice. We next investigated the therapeutic potential of an isoform 1 vector by evaluating its safety profile at high dose in rhesus macaques because of similarities of their immune system and brain structure to humans. First, we generated an AAV-hu68-synapsin-UBE3A-isoform 1 vector. AAVhu68 is also an AAV9 variant developed in-house that works well in nonhuman primates. We injected AAVhu68-hSyn-UBE3A-isoform 1 vector into the cerebrospinal fluid of the cisterna magna of three rhesus macaques (NHP-1, NHP-2, NHP-3) at a high dose of 3×10¹³ GC/animal. After 35 days, macaques were taken down and evaluated for transgene expression, immune response, and adverse effects. No animal presented with a clinically remarkable condition or neurological concern from cage side assessments. Blood chemistry testing indicated normal clotting, liver and kidney function in treated macaques. We observed low levels of isoform 1 vector in several brain regions including the medulla and parietal and temporal cortex and higher levels in the dorsal root ganglia of the spine (FIG. 2A). In addition, we detected low expression of human UBE3A-isoform 1 mRNA in several brain regions including the dorsal root ganglia of the spine (FIGS. 2B and 2C). The localization of engineered human UBE3A isoform 1 transcript was confirmed by fluorescence images of dorsal root ganglia (cervical, thoracic, and dorsal segments) from thee treated NHPs. The UBE3A isoform 1 transcript localization was determined using an RNAscope probe specific for the engineered human sequence (FIGS. 7A to 7I), and counterstained with DAPI (nuclei). Images of regions of interest were taken at various magnifications, and images are presented at 20× magnification. FIGS. 7A to 7I show fluorescent images of engineered human UBE3A isoform 1 (hUBE3A-1) transcript localization in dorsal root ganglia (cervical, thoracic and dorsal segments) from three treated non-human primates (NHPs; in a 35-day study). Treatment comprised administration of AAV-hu68-hSyn-UBE3A-isoform 1 at a dose of 3×10¹³ GC/animal via cisterna magna (ICM) route. Images of regions of interest are presented at 20× magnification. FIG. 7A shows fluorescent image of engineered hUBE3A-1 transcript localization in cervical segment of dorsal root ganglia from NHP-1. FIG. 7B shows fluorescent image of engineered hUBE3A-1 transcript localization in cervical segment of dorsal root ganglia from NHP-2. FIG. 7C shows fluorescent image of engineered hUBE3A-1 transcript localization in cervical segment of dorsal root ganglia from NHP-3. FIG. 7D shows fluorescent image of engineered hUBE3A-1 transcript localization in thoracic segment of dorsal root ganglia from NHP-1. FIG. 7E shows fluorescent image of engineered hUBE3A-1 transcript localization in thoracic segment of dorsal root ganglia from NHP-2. FIG. 7F shows fluorescent image of engineered hUBE3A-1 transcript localization in thoracic segment of dorsal root ganglia from NHP-3. FIG. 7G shows fluorescent image of engineered hUBE3A-1 transcript localization in lumbar segment of dorsal root ganglia from NHP-1. FIG. 7H shows fluorescent image of engineered hUBE3A-1 transcript localization in lumbar segment of dorsal root ganglia from NHP-2. FIG. 7I shows fluorescent image of engineered hUBE3A-1 transcript localization in lumbar segment of dorsal root ganglia from NHP-3.

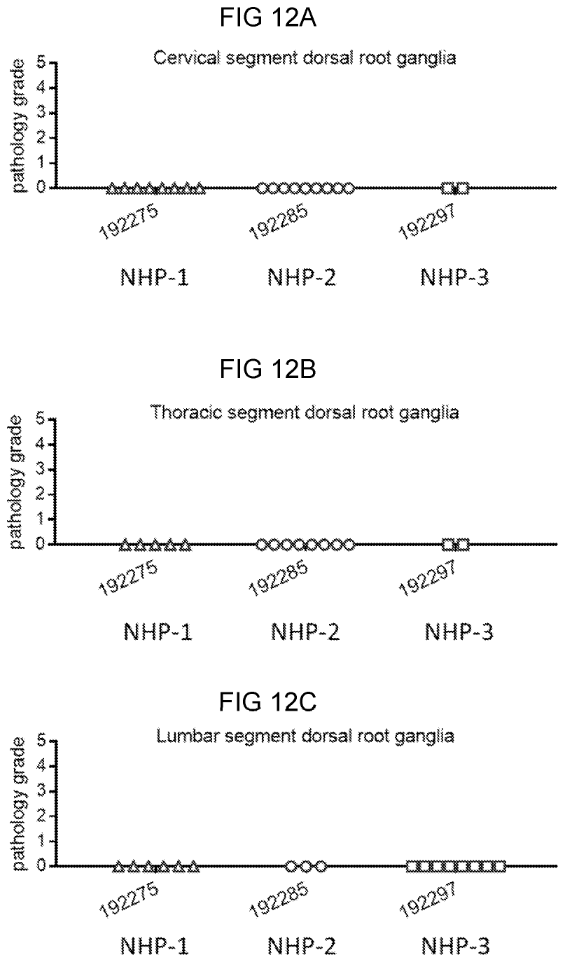

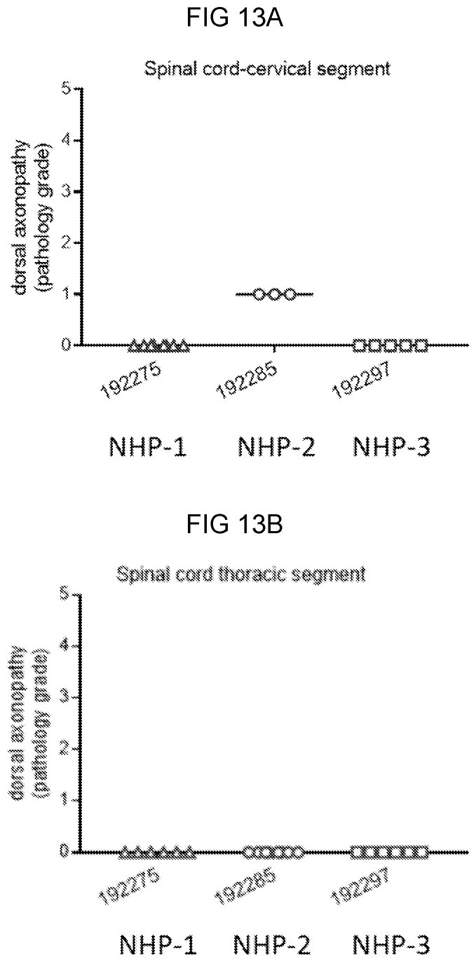

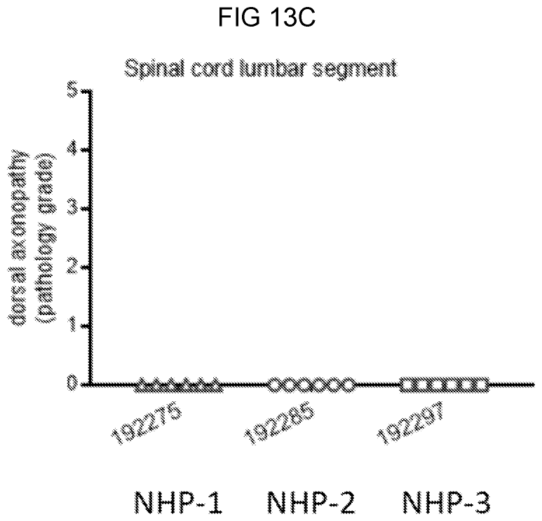

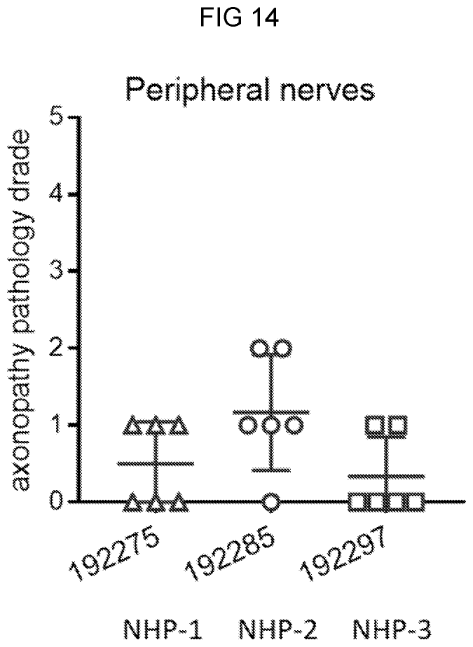

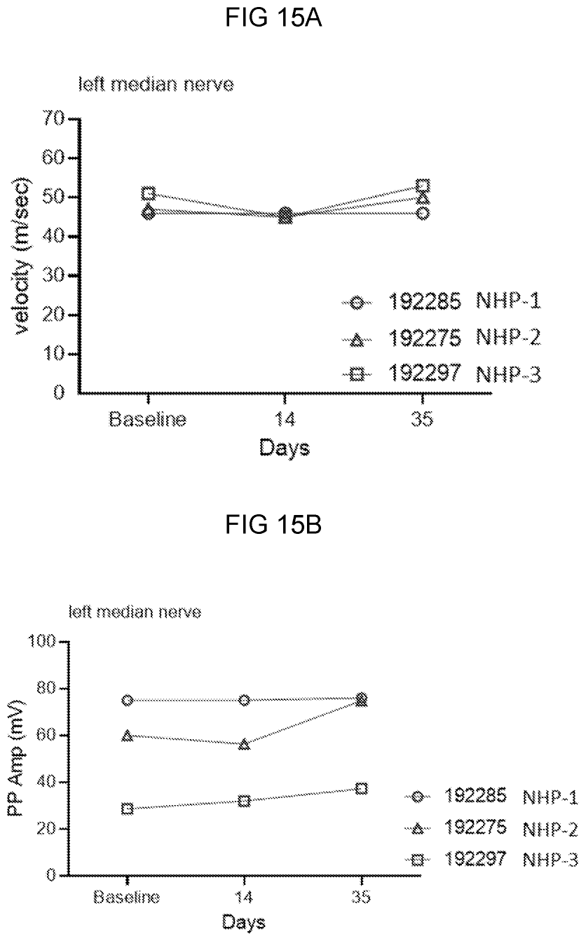

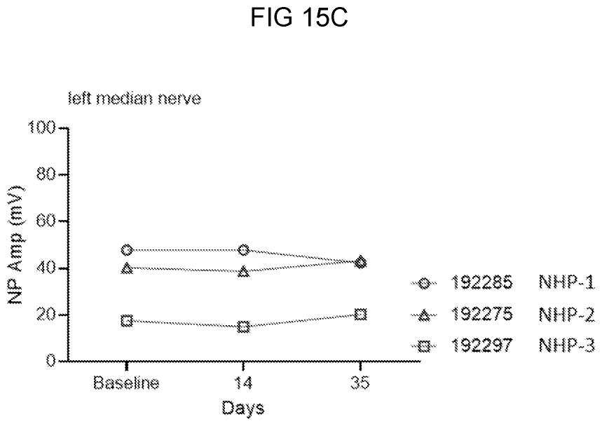

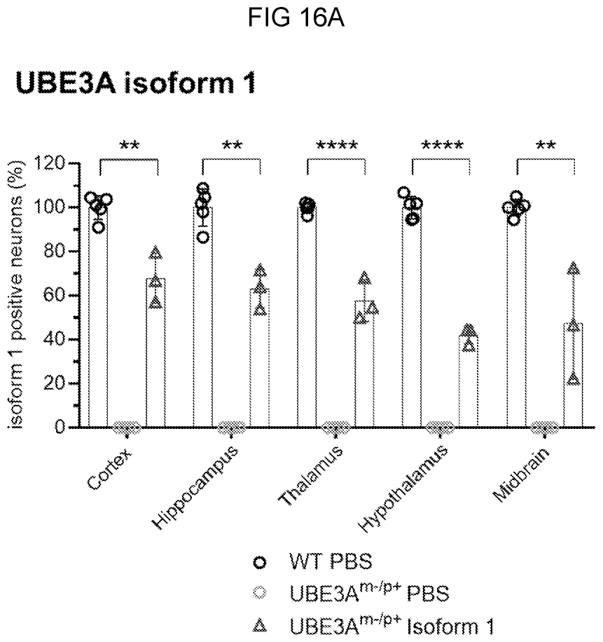

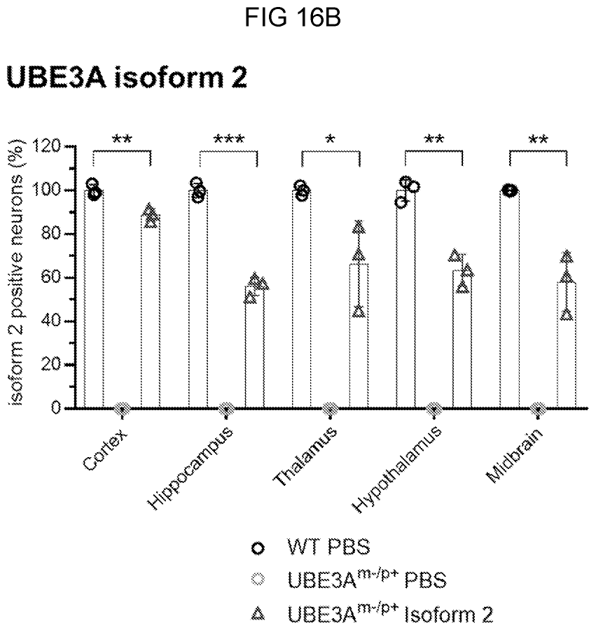

The immune response was minimal with ELISPOT analyses indicating a moderate immune response to hu68 capsid as expected. Finally, dorsal root ganglia (DRG) toxicity is a platform issue of central nervous system directed and high dose AAV in nonhuman primates and potentially humans. However, we observed only mild DRG (FIGS. 12 to 12C) and neuron toxicity in the spinal cord (FIGS. 13A to 13C) and peripheral nerves (FIGS. 13A to 13C) of one animal. In summary, these findings suggest that high dose ICM delivery of an AAVhu68-hSyn-UBE3A-isoforml vector was well-tolerated in nonhuman primates. Additionally, conclusion from this data set is that robust UBE3A isoform 1 transcript localization could be detected in all DRG segments in agreement with pPCR analyses. The absence of significant AAV toxicity in treated NHPs occurs despite robust transgene DRG expression.

### Example 3—NHP Pilot Study Investigating the Safety and Expression of hUBE3A Isoform 1±miR183 Target Sequences in Rhesus Macaques

Because of concerns regarding high dose AAV-induced DRG toxicity in nonhuman primates, and humans, we simultaneously employed a microRNA strategy to downregulate isoform 1 protein expression in the DRG and thus mitigate DRG toxicity. Inclusion of miR183 target sites in the 3′UTR of transgenes (in this case UBE3A-isoform 1) selectively promotes RISC complex-mediated degradation of isoform 1 transgene mRNA in sensory neurons of the DRG, while preserving therapeutic transgene expression in target neurons in the brain. We inserted four copies of a miR183 binding site between the 3′ end of highest expressing engineered isoform 1 or isoform 2 and the SV40 polyadenylation site and packaged it into an AAVhu68 capsid. SEQ ID NO: 11 provides a sequence of one copy of the miRNA183(or miR183) targeting sequence. In this way, we generated an AAV-hu68-synapsin-UBE3A-isoform 1-4×miR183. The AAVhu68 capsid is generated in a packaging host cell using triple transfection techniques in a trans plasmid comprising AAV2 rep coding sequences and the hu68 VP1 coding sequence of SEQ ID NO: 14, co-transfected with the cis plasmid containing the vector genome and a trans plasmid expressing the necessary adenovirus helper functions not provided by the packaging host cell.

Inclusion of 4×miR183 cassette did not significantly affect UBE3A-isoform 1 or isoform 2 protein expression in adult wild type or AS mice injected intravenously at a dose of 1×10¹¹ GC/animal.

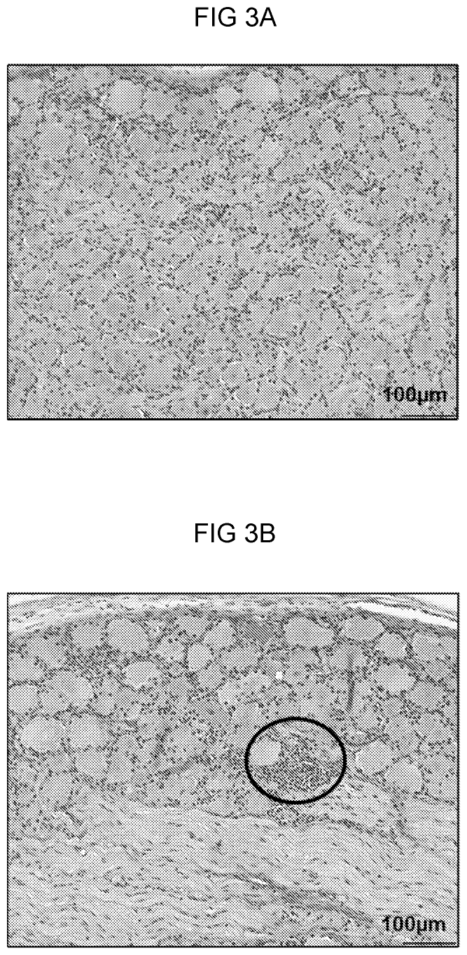

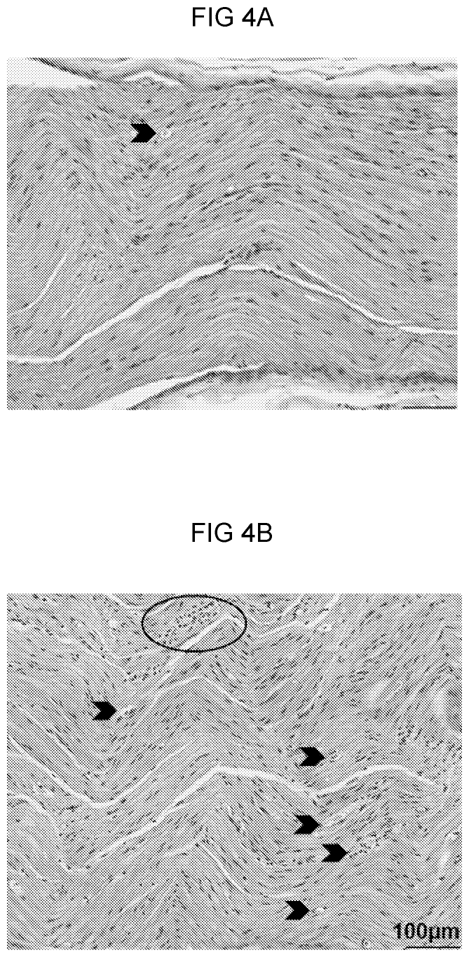

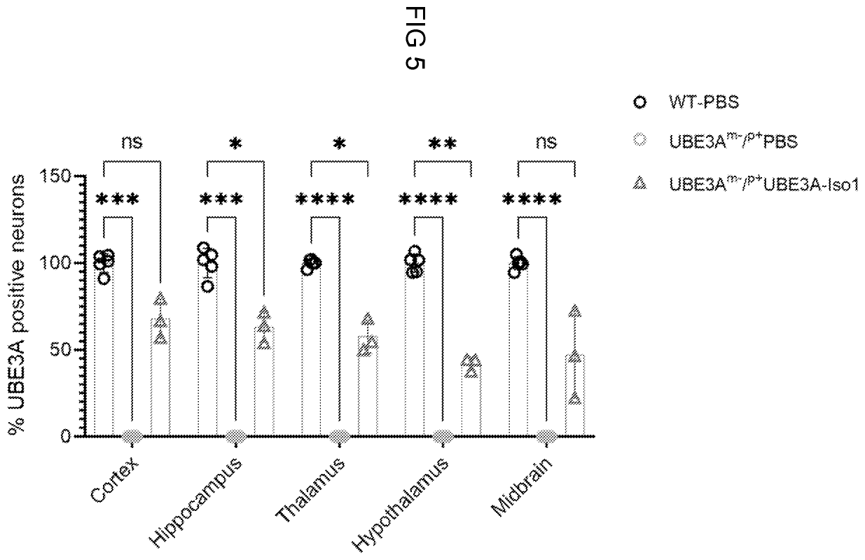

We next compared the safety and expression profile of the rAAVhu68-synapsin-UBE3A-isoform 1-4×miR183 vector with previously analyzed rAAVhu68-synapsin-UBE3A-isoform 1-miR183 vector in rhesus macaques. AAV-hu68-synapsin-UBE3A-isoform 1-4×miR183 was injected at 3×10¹³ GC/animal into the cisterna magna of three macaques and 35 days later macaques were taken down for analysis. All animals were in clinically unremarkable condition for the duration of the study. We observed low levels of isoform 1-miR183 vector in some brain regions such as the medulla with higher levels in dorsal root ganglia and spinal cord as expected. In addition, we detected low expression of human isoform 1-miR183 mRNA in the dorsal root ganglia and spinal cord that was often comparable to isoform 1. Like isoform 1, isoform 1-miR183 was well tolerated in nonhuman primates with minor immunogenicity and no impact on clotting, liver and kidney function. Similarly, we did not observe significant DRG or neuron toxicity in macaques treated with isoform 1-miR183. See, FIGS. 3A-B. The impact of rAAVhu68.synapsin-UBE3A-isoform 1 in peripheral nerve is show in FIGS. 4A-4B.

Vector biodistribution and mRNA expression was evaluated and shown in FIGS. 2A-2C.

In summary, these findings suggest that high dose ICM delivery of an AAVhu68-hSyn-UBE3A-isoforml-miR183 vector was well-tolerated in nonhuman primates. This vector, while not useful in nonhuman primates, could be useful for mitigating DRG and neuronal toxicity if it occurs in human clinical testing.

All documents cited in this specification are incorporated herein by reference. The sequence listing filed herewith named “21-9579PCT_SeqListing_ST25” and the sequences and text therein are incorporated by reference. U.S. Provisional Application No. 63/119,860, filed Dec. 1, 2020, and U.S. Provisional Application No. 63/179,807, filed Apr. 26, 2021 are incorporated herein by reference. While the invention has been described with reference to particular embodiments, it will be appreciated that modifications can be made without departing from the spirit of the invention. Such modifications are intended to fall within the scope of the appended claims.

1. A composition comprising a stock of recombinant adeno-associated virus (rAAV) useful for treatment of Angelman syndrome (AS), the rAAV comprising an AAV capsid and a vector genome packaged therein, said vector genome comprising:
(a) an AAV 5′ inverted terminal repeat (ITR);
(b) a UBE3A nucleic acid sequence comprising SEQ ID NO: 9 or a sequence at least 95% identical thereto encoding UBE3A isoform 1 protein (SEQ ID NO: 2), wherein the nucleic acid sequence is operably linked to regulatory elements which regulate expression of the UBE3A protein in human cells;
(c) regulatory elements which direct expression of the UBE3A of (b); and
(d) an AAV 3′ ITR.

2. The composition according to claim 1, wherein the regulatory elements comprise a neuron-specific promoter

3. The composition according to claim 2, wherein the neuron-specific promoter is a synapsin promoter.

4. The composition according to claim 3, wherein the synapsin promoter is a shortened promoter having the nucleic acid sequence of SEQ ID NO: 12.

5. The composition according to claim 1, wherein the regulatory elements comprise a constitutive promoter.

6. The composition according to claim 1, wherein the regulatory elements further comprise one or more enhancer and one or more introns.

7. The composition according to claim 1, wherein the regulatory sequences further comprise one or more targeting sequences for miR in dorsal root ganglia selected from miR182 and/or miR183, said targeting sequences operably linked to the UBE3A nucleic acid sequence.

8. The composition according to claim 1, wherein the regulatory sequences further comprise one or more targeting sequences for miR in dorsal root ganglia selected from miR182 and/or miR183, said targeting sequences located downstream of the UBE3A nucleic acid sequence.

9. The composition according to claim 1, wherein the regulatory sequences further comprise four targeting sequences for miR183, said targeting sequences located downstream of the UBE3A nucleic acid sequence.

10. The composition according to claim 1, wherein the regulatory sequences comprise four copies of SEQ ID NO: 11.

11. The composition according to claim 1, wherein the AAV capsid is a AAVhu68 capsid.

12. The composition according to claim 1, wherein the AAV capsid is a AAVhu68 capsid generated from expression of the nucleic acid sequence of SEQ ID NO: 14 or SEQ ID NO: 16.

13. The composition according to claim 1, wherein the AAV capsid is a AAVrh91 capsid.

14. The composition according to claim 1, wherein the AAV capsid is a AAVrh91 capsid generated from expression of the nucleic acid sequence of SEQ ID NO: 17 or SEQ ID NO: 19.

15. The composition according to claim 1, which is an aqueous suspension further comprising a physiologically compatible carrier, buffer, adjuvant, and/or diluent.

16-17. (canceled)

18. A method of treating Angelman Syndrome comprising administering to a patient in need thereof the composition of claim 1.

19. A method for treating one or more symptoms of Angelman syndrome in a patient having deficient UBE3A expression in neurons, said method comprising delivering the composition of claim 1.

20. The method according to claim 18, wherein the symptoms are selected from one or more of: delayed development, intellectual disability, severe speech impairment, ataxia and/or epilepsy.

21. The method of claim 18, wherein the composition is delivered intrathecally to the patient.

22. The method according to claim 18, wherein the patient is injected with at least 1×10¹⁰ to 1×10¹³ GC/kg of the rAAV.
